(19) United States
(12) Patent Application Publication
(10) Pub. No.: US 2007/0110707 A1
(43) Pub. Date: May 17, 2007

(54) Method of treating diseases involving non-enzymatic glycation

(75) Inventor: Nathan Ravi, Chesterfield, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Washington University, St. Louis, MO (US)

(21) Appl. No.: 11/555,320

(22) Filed: Nov. 1, 2006

Related U.S. Application Data
(60) Provisional application No. 60/733,820, filed on Nov. 4, 2005.

(51) Int. Cl. A61K 31/785; A61K 33/10; A61K 33/24; A61K 31/433; A61K 31/34

(52) U.S. Cl. 424/78.12; 424/686; 424/687; 424/617; 514/362; 514/471

(57) ABSTRACT

Methods for preventing or diminishing non-enzymatic glycation as well as methods for treating diseases involving non-enzymatic glycation are disclosed. The methods involve administering to a patient an agent that decreases serum inorganic phosphate levels. The agent can be a carbonic anhydrase inhibitor diuretic agent or an agent that binds phosphate in the gastrointestinal tract.

### RELATED APPLICATION

This application claims the priority of provisional application No. 60/733,820 filed Nov. 4, 2005, which is fully incorporated by reference as if fully set forth herein.

### FIELD

This invention relates generally to diseases and conditions involving non-enzymatic glycation and, more particularly, to methods and compositions for treating or preventing non-enzymatic glycation and diseases associated with non-enzymatic glycation.

### BACKGROUND

Amino acids and amino groups of proteins react non-enzymatically with reducing sugars in a process known as non-enzymatic glycation of Maillard reaction (Maillard, C R Acad. Sci 154:66-8, 1912). The products of this reaction are generally referenced as advanced glycation end products (AGE's). The AGE's thus formed can eventually cross link with peptides and proteins to form complexes that can lead to pathologies in AGE-related diseases (for review see Bucala et al., Adv. Pharmacol. 23:1-34, 1992; Brownlee, Metabolism 49:9-13, 2000; Singh et al., Diabetologia 44:129-146, 2001). Elevated amounts of AGE's are believed to be involved in clinically diverse disease processes including diabetic complications (Vlassara et al., Lab. Invest. 70:138-151, 1994; Jakus et al., Physiol Res. 53:131-142, 2004), renal failure (Makita et al, N. Eng. J. Med. 325:836-842, 1991; Schinzel et al. Nephron 87:295-303, 2001), Alzheimers disease (Munch et al., Brain Res. Rev. 23:134-143, 1997; Munch et al., Neural. Transm. 105: 439-51, 1998), Pick's disease (Kimura et al., Neurosci. Lett. 219:95-8, 1996; Susaki et al., Am. J. Pathol. 153:1149-55, 1998), and age-related diseases including decreased skin elasticity (Sell et al., J. Clin. Invest 85:380-4, 1990), male erectile dysfunction (Jiaan et al., World J. Urol. 13:369-75, 1995), pulmonary fibrosis (Silacci, J. Hypertension 20:1483-85, 2002), atherosclerosis (Stitt et al., Ann. N.Y. Acad. Sci. 811:115-29, 1997) and ocular diseases (Stitt, Br. J. Ophthalmol. 85: 746-753).

The levels of non-enzymatic glycation products have been shown to be related to blood glucose levels in diabetic patients (Koenig et al., N. Engl. J. Med. 295:417-420, 1976). Factors other than glucose have also been reported to influence non-enzymatic glycation including oxygen tension and 2,3-diphosphoglycerate concentrations (Smith et al., J. Clin. Invest. 69:1164-1168, 1982). Increasing phosphate concentrations have also been reported to enhance glycation in vitro (Hall et al. Biochimica et Biophysica Acta 993:217-223, 1989; Kunika et al., Life Sci. 45:623-630, 1989; Kunika et al., Diabetes Research and Clinical Practice 17:9-16, 1992). In addition, in a non-diabetic healthy humans, inorganic phosphate concentrations have been reported to be significantly correlated to the levels of the glycation product, fructosamine, corrected for glucose concentrations. (Kunika, supra, 1992). Nevertheless, no treatment approach for decreasing non-enzymatic glycation through modification of inorganic phosphate levels has been heretofore suggested.

### SUMMARY

Accordingly, the present inventor has succeeded in devising an new approach for treating, preventing or diminishing the magnitude of non-enzymatic glycation by decreasing the plasma levels of inorganic phosphate in a patient in need thereof. The reduction in inorganic phosphate levels can be from hyperphosphatemia levels of greater than about 5 mg/dL or greater than about 6 mg/dL or from normal phosphatemia levels of less than about 5 mg/dL down to levels of not less than about 2 mg/dL.

Thus, in various embodiments, the present invention is directed to a method of treating a disease involving non-enzymatic glycation. The method can comprise administering to a patient in need thereof an effective amount of at least one agent that decreases serum phosphate concentration, wherein prior to treatment the patient has a serum phosphate concentration of not more than about 5.0 mg/dL. In various aspects of the present invention, the method can also involve selecting at least one agent on the basis of the agent being effective in decreasing serum phosphate concentration and administering an effective amount of the at least one agent to a patient in need thereof. The reduction in phosphate concentration can be to levels which do not produce undesirable side effects due to hypophosphatemia such as, for example, not less than about 2 mg/dL.

In various embodiments, the invention can involve a method for treating a disease involving non-enzymatic glycation in which the disease is one other than end stage renal disease. The method can comprise administering to a patient in need thereof an effective amount of at least one agent that decreases serum phosphate concentration. In various aspects of the present invention, the method can also involve selecting at least one agent on the basis of the agent being effective in decreasing serum phosphate concentration and administering an effective amount of the at least one agent to a patient in need thereof. The reduction in phosphate concentration can be to levels which do not produce undesirable side effects due to hypophosphatemia such as, for example, not less than about 2 mg/dL.

The present invention, in various embodiments, can also involve preventing or diminishing non-enzymatic glycation in a patient. The method can comprise administering to a patient in need thereof, an effective amount of at least one agent that decreases serum phosphate concentration, wherein prior to administering the agent, the patient has a serum phosphate concentration of not more than about 5.0 mg/dL. In various aspects of the present invention, the method can also involve selecting at least one agent on the basis of the agent being effective in decreasing serum phosphate concentration and administering an effective amount of the at least one agent to a patient in need thereof. The reduction in phosphate concentration can be to levels which do not produce undesirable side effects due to hypophosphatemia such as, for example, not less than about 2 mg/dL.

In various embodiments, the agent that decreases serum phosphate concentration can be a diuretic and in particular a carbonic anhydrase inhibitory diuretic or a phosphate binder or a combination thereof. Examples of diuretics that have carbonic anhydrase inhibitory activity include acetazolamide, dichlorphenamide, and methazolamide as well as furosemide. Phosphate binders include aluminum hydroxide, calcium salts, such as calcium acetate, calcium carbonate or calcium gluconate, magnesium compounds such as magnesium hydroxide or magnesium carbonate, lanthanum carbonate or phosphate binding cationic polymers such as sevelamer hydrochloride. The present invention also includes the administration of combinations of diuretics, combinations of calcium binders or combinations of one or more diuretics and one or more calcium binders.

Among the various diseases involving non-enzymatic glycation that can be treated by the methods of the present invention, the patient can suffer from diseases such as, for example, diabetes and diabetic complications, renal failure in both diabetic and non-diabetic patients, Alzheimers disease, Pick's disease and age-related diseases including decreased skin elasticity, male erectile dysfunction, pulmonary fibrosis, atherosclerosis and ocular diseases as well as disease in which increased levels of glycation products constitutes a risk factor for mortality and other adverse cardiovascular events following acute myocardial infarction following percutaneous coronary intervention.

In various embodiments, the agent that decreases serum phosphate concentration can be administered in combination with an effective amount of at least one agent that decreases blood glucose levels, wherein the at least one agent that decreases blood glucose levels is insulin, a sulfonylurea, a biguanide compound, a meglitinide compound or a compound that acts upon glucose digestion or metabolism second agent that decreases blood glucose levels.

In various embodiments, the present invention can also include a pharmaceutical composition for treating a disease involving non-enzymatic glycation. The composition can comprise an effective amount of at least one agent that decreases serum phosphate concentration and an effective amount of at least one agent that decreases serum glucose levels.

In various embodiments, the present invention can also comprise a kit for treating a disease involving non-enzymatic glycation. The kit can comprise an effective amount of at least one agent that decreases serum phosphate concentration and an effective amount of at least one agent that decreases serum glucose levels packaged separately in a container.

### BRIEF DESCRIPTION OF THE DRAWINGS

FIG. 1 illustrates (A) the normal % probability plot of factors that influence pH, (B) the expected random distribution of Studentized residuals on a normal plot and (C) the interactions of oxygen with phosphate and ribose shown as a cubic plot.

FIG. 2 illustrates (A) the half-normal % probability plot of the various factors and their two-factor interactions on the amount of protons generated, (B) the main effect of phosphate, oxygen, and ribose along with their two and 3-factor interaction shown as a cubic plot, and (C) a combined contour and surface plot of the interaction between phosphate and ribose, mathematically computed, with pO₂ and EDTA at the “high level” and the rest of the factors at the “low level.”

FIG. 3 illustrates the two-factor interaction plots between phosphate and (A) oxygen, (B) light, and (C) EDTA.

FIG. 4 illustrates (A) the normal % probability plot of the Studentized residuals; (B) the effect of all the significant variables a composite graph and (C) the interaction between phosphate and light.

FIG. 5 illustrates a three-dimensional plot of values obtained from patients in which the x-axis and y-axis represent average phosphate and glucose level over a four-month period and the z-axis represents the HbA1c values.

### DETAILED DESCRIPTION

The present invention involves methods and compositions for treating, preventing or diminishing the magnitude of non-enzymatic glycation in diseases involving non-enzymatic glycation by decreasing the plasma levels of inorganic phosphate in a patient in need thereof

As discussed above, diseases and disease processes involving non-enzymatic glycation are diverse and examples of some of such diseases and disease processes include diabetes and diabetic complications, renal failure in both diabetic and non-diabetic patients, Alzheimers disease, Pick's disease and age-related diseases including decreased skin elasticity, male erectile dysfunction, pulmonary fibrosis, atherosclerosis and ocular diseases. Patients having such diseases can benefit by reduction of inorganic phosphate levels according to the methods of the present invention.

Non-enzymatic glycation can also be assessed in patients by measuring levels of glycosylated proteins using methods known in the art. For example, HbA1c has been shown to be formed by the direct reaction of hemoglobin with glucose and it has been suggested that this can serve as a model system for non-enzymatic glycosylation of protein. (McDonald et al., J. Biol. Chem. 253:2327-2332, 1978). This test has been particularly useful in monitoring glycemia control in diabetes (Cerami et al., Metabolism 28 (Suppl):431-437, 1979).

In other diseases, increased levels of glycation have been shown to constitute a risk factor for subsequent pathologic conditions. For example, high HbA1c values have been shown to be an independent risk factor for death following acute myocardial infarction (Chowdhury et al., Postgrad. Med. J. 74:480-1, 1998) and for adverse cardiovascular events including mortality following percutaneous coronary intervention (Corpus et al., Am J. Cardiol. 92:1282-6, 2003; Corpus et al., J. Am Coll. Cardiol. 43:8-14, 2004). In one such study, it was shown that HbA1c levels in the high normal range of 6% to 7% in nondiabetic patients was a significant independent risk factor for adverse cardiovascular events (Corpus et al., supra, 2003).

In addition to the HbA1c method, the measurement of fructosamine levels have also been suggested to provide a measure of glycation (Kunika et al., supra, 1992).

Thus, in various embodiments, the methods of the present invention can be applicable to diseases known to be associated with non-enzymatic glycation and/or patients showing high levels of glycation such as can be assessed through measuring HbA1c levels or by any other method known in the art. In addition, in various embodiments, the methods of the present invention can be applicable to individuals that have glycation levels in the normal range, i.e. less than about 6%. For example, in patients having a disease associated with non-enzymatic glycation or having a condition associated with a risk of adverse events associated with glycation, inorganic phosphate levels can be decreased to a hypophosphatemia level of about 2 mg/dL.

In various embodiments, the methods of the present invention can involve decreasing serum levels of inorganic phosphate. Reference herein to serum levels of inorganic phosphate or to serum concentrations of inorganic phosphate are intended to be interchangeable and such references are intended to include levels or concentrations of inorganic phosphate measured in serum, plasma, whole blood or any fraction thereof as well as any indirect estimation of serum inorganic phosphate levels from levels or concentrations in any other sample obtained from the patient. Serum inorganic phosphate levels are usually measured spectrophotometrically as the complex of phosphate ions with ammonium molybdate and levels are usually expressed in terms of phosphorus content (see, for example Cogan et al., Anal. Biochem. 271:29-35, 1999 which is incorporated by reference). Normal levels of serum inorganic phosphate in humans range from about 2.5 to about 4.5 mg/dL in adults and from about 3 to about 6 mg/dL in children.

Decreasing serum levels of inorganic phosphate can be achieved by different approaches. For example, diuretics such as, for example carbonic anhydrase inhibitors can increase renal excretion of inorganic phosphate and thereby decrease serum phosphate levels. Such carbonic anhydrase inhibitor diuretic agents include acetazolamide, dichlorphenamide, methazolamide. In addition, other classes of diuretic agents can also exhibit carbonic anhydrase activity and, as a result, increase renal excretion of inorganic phosphate. Such agents include some of the sulfonamide-based loop diuretics such as, for example, furosamide.

Inorganic phosphate levels in the serum can also be decreased by oral administration of phosphate binding agents. Phosphate binding agents combine with phosphate in the gastrointestinal tract to form a complex that is not absorbed into the blood stream. Phosphate binding agents can include, for example, aluminum hydroxide, calcium salts, such as calcium acetate, calcium carbonate or calcium gluconate, magnesium compounds such as magnesium hydroxide, magnesium acetate, magnesium carbonate or magnesium gluconate, lanthanum carbonate or phosphate binding cationic polymers such as sevelamer hydrochloride or combinations thereof. Phosphate binding cationic polymers are described in U.S. Pat. No. 5,496,545 which is incorporated by reference.

In various embodiments, the agent that decreases serum inorganic phosphorus levels can be administered to a patient in an amount effective in reducing phosphorus levels. As used herein, the term treatment is intended to include preventing the appearance of a disease or condition, diminishing the severity of a disease or condition or alleviating or diminishing certain aspects or symptoms of a disease or condition.

The methods of the present invention are useful in treating mammals. Such mammals include humans as well as non-human mammals. Non-human mammals include, for example, companion animals such as dogs and cats, agricultural animals such live stock including cows, horses and the like, and exotic animals, such as zoo animals.

The agents that decrease serum inorganic phosphate concentrations in accordance with the methods of the present invention, can be administered in combination with agents that decrease serum glucose concentrations. Reference herein to serum levels of glucose or to serum concentrations of glucose are intended to be interchangeable and such references are intended to include levels or concentrations of glucose measured in serum, plasma, whole blood or any fraction thereof as well as any indirect estimation of serum glucose levels from levels or concentrations in any other sample obtained from the patient. Glucose levels are usually measured by enzymatic assay with photometric detection. Typical enzymatic serum glucose assays can be base upon glucose oxidase or hexokinase (see for example, Giampietro et al., Clin. Chem. 28:2405-7, 1982, which are incorporated by reference). Normal levels for serum glucose can be less than about 110 mg/dL.

Agents that can be administered to decrease serum glucose concentrations include insulin which can be administered parenterally or a hypoglycemic agent which can be administered orally to achieve glycemia control. Oral hypoglycemic agents include sulfonylureas such as acetohexamide, chlorpropanamide, tolazamide, tolbutamide, glyburide, glipizide, and glibomuride; biguanides such as metformin and buformin; or meglitinide compounds such as repaglinide and nateglinide. In such patients, additional administration of at least one agent that decreases serum inorganic phosphate concentration advantageously minimize the generation of non-enzymatic glycation products. Such combination treatment is believed to diminish the severity of conditions arising from increased glycation, thereby, slowing the progression of diabetic complications. Other hypoglycemic agents that act on carbohydrate metabolism or on carbohydrate metabolism can also be administered (see for example, U.S. Pat. No. 6,809,115; U.S. Pat. No. 6,821,977; Murai et al., Life Sci. 71:1405-15, 2002). It is also possible that controlling glycemia levels can be advantageous in nondiabetic patients having high serum levels of glycation products, such as HbA1c levels exceeding 6%. Accordingly the abovementioned agents that decrease serum glucose levels can in certain embodiments be administered with agents that decrease serum inorganic phosphorus levels.

The combination treatment can involve co-administration of an agent that decreases serum inorganic phosphate concentration and an agent that decreases serum glucose concentration, i.e. at the same time and by the same or different routes of administration, depending upon the particular agents used. The combination treatment can also involve administering the agents at different times and on different schedules.

In various embodiments, the present invention can comprise pharmaceutical compositions for treating a disease involving non-enzymatic glycation, the compositions comprising an effective amount of at least one agent that decreases serum phosphate concentration and an effective amount of at least one agent that decreases serum glucose levels. The pharmaceutical composition is in a formulation suitable for administration by an appropriate route for the combination of agents. For example, for pharmaceutical compositions including insulin as the agent that decreases serum glucose levels in the combination of agents, a parenteral formulation can be used. Further, for pharmaceutical compositions including oral hypoglycemic agents in the combination of agents, an oral route of administration can be used.

In various embodiments, the present invention can also comprise a kit for treating a disease involving non-enzymatic glycation. The kit can comprise an effective amount of at least one agent that decreases serum phosphate concentration and an effective amount of at least one agent that decreases serum glucose levels packaged separately in a container. In various embodiments, the separate packaging allows the at least one agent that decreases serum phosphate concentration and the at least one agent that decreases serum glucose levels to be formulated separately. This can allow one of the agents to be formulated in a parenteral formulation and the other to be formulated in a separate oral formulation. Thus, for example, an orally active agent that decreases serum phosphate concentration can be in a formulation for oral administration and a parenterally active agent, such as insulin, can be in a separate formulation for parenteral administration. The kits can also contain both agents formulated separately in oral formulations or both agents formulated separately in parenteral formulations, depending upon the oral or parenteral activity of the particular agents. The separate packaging can also provide separate doses for administering the agent that decreases serum phosphate concentration and the agent that decreases serum glucose levels according to a different schedule of administration.

The agent that decreases serum phosphate concentration and the agent that decreases serum glucose levels can be administered by any suitable route of administration known in the art such as, for example, buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, oral, otic, biliary perfusion, cardiac perfusion, priodontal, rectal, spinal, subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

In some instances, the oral route of administration can particularly advantageous for administration of agents in accordance with the present invention. Phosphate binding agents, in particular, are intended to be administered orally, usually with meals. The orally administered agents can be can be encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The invention can be further understood by reference to the examples which follow.

### EXAMPLE 1

This example illustrates the identification of factors influencing non-enzymatic glycation. The reaction variables studied were phosphate, HEPES, Tris, oxygen, EDTA, light, shaker and excess ribose concentration.

To identify factors involved in non-enzymatic glycation, we performed a fractional factorial experiment in which a model reaction between CBZ-lysine and ribose was investigated in vitro at 37° C. and pH 7.4. The reaction variables were phosphate, HEPES, Tris, oxygen, EDTA, light, shaker, and excess ribose; the responses measured were for changes in pH, pO₂. pCO₂, fluorescence, and CBZ-L-lysine, and formation of products P1, P2, and P3, as identified on HPLC.

The Na-CBZ-L-lysine-ribose reaction system is based upon Na-CBZ-L-lysine which has a carbo-benzoyl (CBZ) group attached to the alpha amino group of L-lysine. Introduction of CBZ facilitates detection by a UV detector; Na-CBZ-L-lysine is more hydrophobic than lysine and facilitates separation by reverse phase HPLC; and has only the epsilon amino group for reaction. We chose ribose as the reducing sugar for its faster reactivity than D-glucose thereby decreasing the experimental time. The second aspect of our second approach was to use a fractional factorial design. Use of statistical screening designs is often very valuable in investigating complex process particularly the Maillard reaction. We studied the reaction between ribose and Na-CBZ-L-lysine under various reaction conditions. The experimental variables were phosphate buffer, Tris buffer, HEPES buffer, ambient laboratory light, air, ethylenediamine tetra-acetic acid (EDTA), and incubation shaker speed. We monitored the reaction for changes in pH, pO₂, pCO₂, osmolarity, fluorescence, color formation, consumption of CBZ-lysine, and formation of products as detected on high pressure liquid chromatography (HPLC).

Materials & Methods:

Statistical design: Eight experimental variables investigated were phosphate (A), Tris (B), HEPES (C), oxygen (D), ambient laboratory light (E), shaker speed (F), EDTA (G), and ribose (H). We evaluated each variable at two levels. The full factorial design for eight variables, each at two levels, requires a total of 2⁸ or 256 runs. However, as we were interested, initially, in determining only the main effect and their two-factor interactions, we used a 1/16 fraction of the original 256 run full factorial design. This design was obtained by first writing a full factorial design for three factors in the standard format (see Table I, first three columns A, B, and C, and the top eight rows).

TABLE 1
FRACTIONAL FACTORIAL DESIGN MATRIX

| RUN | PBS | TRIS | HEPES | 02 | Light | Shaker | EDTA | Ribose |
|---|---|---|---|---|---|---|---|---|
| 1 | − | − | − | + | + | + | − | + |
| 2 | + | − | − | − | − | + | + | + |
| 3 | − | + | − | − | + | − | + | + |
| 4 | + | + | − | + | − | − | − | + |
| 5 | − | − | + | + | − | − | + | + |
| 6 | + | − | + | − | + | − | − | + |
| 7 | − | + | + | − | − | + | − | + |
| 8 | + | + | + | + | + | + | + | + |
| 9 | + | + | + | − | − | − | + | − |
| 10 | − | + | + | + | + | − | − | − |
| 11 | + | − | + | + | − | + | − | − |
| 12 | − | − | + | − | + | + | + | − |
| 13 | + | + | − | − | + | + | − | − |
| 14 | − | + | − | + | − | + | + | − |
| 15 | + | − | − | + | + | − | + | − |
| 16 | − | − | − | − | − | − | − | − |

As per convention the negative sign (−) indicates lower limit and the positive sign (+) the upper limit of the variable PBS (0-100 mM), HEPES (0 to 100 mM), TRIS (0 to 100 mM), Oxygen (0, from N₂ flush of solution to atmosphere), Light (absent or ambient), Shaker bath speed (zero or 30 rpm), EDTA (0 to 5 mM), Ribose (50 to 100 mM).

Column D was generated from AB, a product of column A and B. The columns D through H were similarly generated as follows: E=BCD, F=ACD, G=ABC, and H=ABD as shown in Table IA.

TABLE 1A
8 Factors: A, B, C, D, E, F, G, H
Factor Generator
E − BCD; F − ACD; G − ABC, H − ABD
Factorial Effects Defining Contrast
I − ABCG = ABDH = ABEF = ACDF = ACEH = ADEG = AFGH =
BCDE = BCFH = BDFG = BEGH = CDGH = CEFG = DEFH =
ABCDEFGH
Factorial Effects Aliases
[Est. Terms] Aliased Terms
[Intercept] = Intercept
Main factor confounded with three factor interactions
[A] = A + BCG + BDH + BEF + CDF + CEH + DEG + FGH
[B] = B − ACG + ADH + AEF + CDE + CFH + DFG + EGH
[C] = C − ABG + ADE + AEH + BDE + BFH + DGH + EFG
[D] = D + ABH + ACF + AEG + BCE + BFG + CGH + EFH
[E] = E + ABE + ACH + ADG + BCD + BGH + CFG + DFH
[F] = F − ABE + ACD + AGH + BCH + BDG + CEG + DEH
[G] = G + ABC + ADE + AFH + BDF + BEH + CDH + CEF
[H] = H + ABD + ACE + AFG + BCF + BEG + CDG + DEF
Two factor interactions confounded with other two factor interactions
[AB] = AB + CG + DH + EF
[AC] = AC + BG + DF + EH
[AD] = AD + BH + CF + EG
[AE] = AE + BF + CH + DG
[AF] = AF + BE + CD + GH
[AG] = AG + BC + DE + FH
[AH] = AH + BD + CE + FG

The remaining eight runs (rows) for all the columns were obtained by “folding over” that is, in the second set of eight rows the signs were all reversed. This resulted in a resolution IV fractional factorial design as shown in Table I. The factorial effects aliases for the main and two factor interactions are shown in Table IA.

The high and low level, indicated by + and − sign respectively, of each variable is shown in Table II.

TABLE II
VALUES OF CODED VARIABLES

| | LOW LEVEL | HIGH LEVEL |
|---|---|---|
| FACTORS | (−) | (+) |
| Phosphates (mM) | 0 | 100 |
| Tris (mM) | 0 | 100 |
| HEPPES (mM) | 0 | 100 |
| Oxygen (pO2) | N₂ flush | Air |
| Light (for 8 Hrs) | 0 | |
| Shaker (rpm) | 0 | 30 |
| EDTA (mM) | 0 | 5 |
| Ribose (mM) | 50 | 100 |

The responses measured were: 1) change in pH, 2) generation of protons, 3) consumption of oxygen, 4) production of carbon dioxide, 5) changes in the relative intensity of fluorescence, 6) consumption of CBZ-lysine, and 7) formation of products P1, P2, and P3 on HPLC analysis. Care was taken to perform the sixteen experiments were performed in random order. Analysis of data required no transformations. The difference between the average responses when the factor is present and absent gives the average effect of each factor over all conditions of the other variables. Due to the general symmetry of the experimental design matrix, there exists a set of eight measures for each response with and without the factor, Thus, the precision of each effect equals to an eight-fold replication over the entire design space. The complete design was once again replicated in random order about six weeks later. Statistical analysis was performed on the complete data that is, the original as well as the replicates. This allowed for the calculation of the pure error terms and had 32 degrees of freedom. Sixteen statistical parameters consisting of one grand mean, 8 main effects each confounded with four three factor interactions, and 7 two factor interactions each confounded with three other two factor interactions were obtained. The half-normal % probability or the full-normal % probability versus effect shows the effect of each variable on the response. Effects that significantly influence the response are away from the normal distribution line and from the 0.0 axis. We developed a statistical linear model by using only significant variables. The statistical parameters of the model were checked for hierarchy prior to analyzing the experimental variance (ANOVA). The sum of squares (SS), degrees of freedom (DF), Mean Square, F value, and probability>F were obtained for the model. Also, various summary statistics for the model, such as square root of the mean square error (Root MSE), R-Squared, adjusted R-squared (Adj. R-Squared), predicted k-squared (Pred. R-Squared), coefficient of variation (C.V.), and predicted residual sum of squares (PRESS) were calculated. We performed post-ANOVA diagnostic checks including scedacity of the variance. Insignificant main or two-factor interactions were deleted until the Studentized residuals were randomly distributed on a normal probability plot (homoscedastic). The responses were also analyzed for any linear or nonlinear correlation between each other and summarized as a correlation matrix consisting of r-squared valves. R-squared valves greater than 0.5 were considered to be significant.
Methods:

All materials used in the study were obtained from Sigma Corp. (St. Louis, Mo.). The buffers were prepared with double distilled water. Eight different experimental variables (continuous) were evaluated. The runs were made in random order (3, 1, 11, 5, 8, 4, 12, 5, 10, 2, 7, 9, 6, 14, 16, and 13). Two ml of each solution was prepared and pH was adjusted to 7.4. Solutions were filtered through 0.22-micron filters, divided into each of two sterile 10-ml serum vials, and sealed with an airtight rubber septum and aluminum cap. Thus each solution was 1 ml and was contained in a 10-ml serum vial. Solutions which needed to have low O₂ had sterile-filtered nitrogen gas bubbled through the solution for 5 minutes. All solutions were placed in a water bath at 37° C. for a period of one week; samples 1, 2, 7, 8, 11, 12, 13, and 14 were continuously shaken at 30 rpm while the remaining were kept stationary. Eight samples: 1, 3, 6, 8, 10, 12, 13, and 15 were wrapped with aluminum foil to minimize exposure to ambient laboratory light. Samples were checked visually as well as plated on agar culture plates to check for microbial growth. An aliquot of the reaction mixture was withdrawn from the serum vial without introducing any air and it was injected into the blood gas analyzer (Blood gas Corning 170 pH/blood gas analyzer), and the pH, PO₂, and pCO₂ recorded. Air was saturated with water vapor at ambient barometric pressure. The pH of the sample was also measured using a stand-alone pH meter (Fisher Accumet model 25). Fluorescence absorption measurement was made by diluting (10-50 fold) the sample with phosphate A C buffered solution (pH 7.4) using an excitation wavelength of 370 nm and measuring the emission at 430 nm (Perkin-Elmer LS-SD Luminescence Spectrometer). The HPLC condition for analysis of samples was as follows: c-IS stationary phase of 5.0 um particle size packed in a column of diameter 4.6 mm and length of 250 mm. The mobile phase was a mixture of acetonitrile (90% containing 0.1% trifloroacetic acid) and water (10%). The products in the eluent were detected by monitoring its absorbency at 254 nm and 295 nm using a Waters 490E-programmable multiwavelength detector. As the samples had different buffering capacity, depending on the combination of buffers and reactants, the amount of H⁺ needed for a unit change of pH was different for each sample combination. Therefore, a calibration curve was obtained for each combination of buffer solution by titrating with 0.1 N HCl and noting the change in pH.

We determined the trace metal content of the reagents using ICP-MS technique. Typically 0.1 gm of the reagent was dissolved in 100 ml of 1% HNO₃ acid. An internal standard of In (10 ppb) was added and the samples were analyzed a series of certified reference standards and heated to evaporation. The residue was redissolved in water and an aliquot was injected into the ICP-MS. The spectrum was analyzed for Cu, Fe, Cr, Zn, Mn, V, Ni, and Co, based on known internal standards. Samples were run in triplicates.

Results:

As our experiment had eight variables and nine responses, we discuss the effect of all the variables on each response as well as the effect of each variable on all the responses.

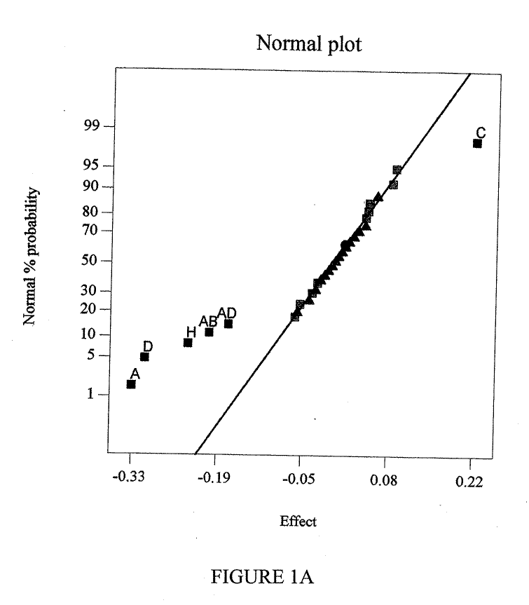

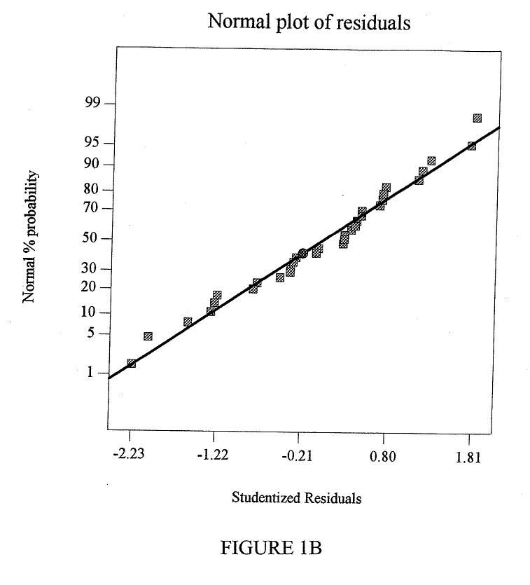

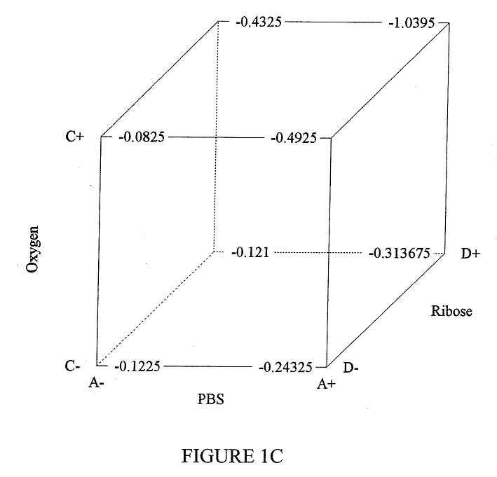

Effect of variables on change in pH: Maximum and minimum decrease in pH was observed in run #4 (ΔpH=−1.23) and #12 (ΔpH=−0.02) respectively. FIG. 1A is a normal % probability plot of factors that influence pH. Main factors A, C, D, G, and H were significant in changing the pH with factor C increasing the pH. Among the two-factor interactions AB, AD, AF were found to be significant. The statistical significance of the linear model and for the factors is shown in Table IA. The effect of AB and AF were confounded by DH and CD. To confirm that phosphate, HEPES, oxygen, EDTA, and ribose were responsible for the effect and not their confounding terms (see Table IA) the insignificant factors light, shaker, and Tris, in this case, were deleted. The data was reanalyzed with the remaining factors as a half-factorial design with less confounding terms. Phosphate, oxygen, ribose, HEPES, and EDTA were observed to be unambiguously significant and in decreasing order. HEPES and EDTA increased the pH. The two-factor interaction was confirmed to arise from interactions between phosphate-oxygen, oxygen ribose, and oxygen-HEPES. The three and four factor interactions were insignificant. The p>F and R-Sqd. value for the half-factorial model were 1.13E-10 and 0.92 respectively and the post-ANOVA statistics were very good with the expected random distribution of Studentized residuals on a normal plot shown in FIG. 1B. The final equation in terms of the coded factors that were significant is shown below as well as in Table III, column 2.
ΔpH=−0.36−0.17*PBS−0.16*pO₂−0.12*ribose+0.11*HEPES+0.047*EDTA−0.09*PBS* pO₂−0.10*pO₂*ribose+0.044*HEPES*PO₂ (1)

TABLE III
STATISTICAL SIGNIFICANCE FOR THE LINEAR MODEL (P > F) AND FOR THE
FACTORS (P > T) FOR EACH OF THE RESPONSES

| | | | | | | CBZ- | | | |
|---|---|---|---|---|---|---|---|---|---|
| | pH | H+ | PO2 | PCO2 | Fluorescence | Lysine | P1 | P2 | P3 |
| Model | 2.08E−13 | 1.84E−18 | 1.33E−05 | 8.47E−12 | 4.28E−19 | 4.45E−04 | 1.51E−02 | 1.017E−02 | 6.36E−02 |
| R-sqd. | 0:9576 | 0.9802 | 0.8559 | 0.9460 | 0.9820 | 0.8531 | 0.7455 | 0.7592 | 0.6712 |
| A: PBS | 4.13E−08 | 1.67E−12 | 9.53E−07 | 2.11E−07 | 2.88E−14 | 2.22E−06 | 3.83E−05 | 1.04E−02 | 1.10E−02 |
| B: TRIS | 3.75E−01 | 5.41E−06 | 4.06E−02 | 5.01E−01 | 3.9E−04 | 2.21E−01 | 3.72E−01 | 4.04E−02 | 6.53E−01 |
| C: HEPPES | 7.08E−06 | 4.79E−02 | 7.04E−01 | 4.73E−01 | 2.67E−03 | 5.03E−02 | 7.94E−01 | 4.71E−01 | 2.12E−01 |
| D: Oxygen | 1.01E−07 | 3.70E−09 | 2.73E−03 | 1.04E−07 | 6.93E−04 | 1.01E−02 | 9.84E−02 | 3.61E−03 | 3.00E−03 |
| E: Light | 2.65E−01 | 4.82E−01 | 1.45E−01 | 3..51E−02 | 5.62E−04 | 2.24E−01 | 2.25E−01 | 7.34E−02 | 5.49E−01 |
| F: Shaker | 1.66E−01 | 1.34E−01 | 4.58E−01 | 3.41E−01 | 1.23E−02 | 8.69E−02 | 6.49E−01 | 4.08E−01 | 6.95E−01 |
| G: EDTA | 1.47E−02 | 4.35E−01 | 3.86E−02 | 6.42E−03 | 2.40E−04 | 1.17E−01 | 2.47E−01 | 7.47E−01 | 7.76E−01 |
| H: Ribose | 2.74E−06 | 3.60E−07 | 2.97E−02 | 3.95E−01 | 5.10E−06 | 1.40E−02 | 5.04E−01 | 1.04E−01 | 9.04E−01 |
| 11 | 1.71E−05 | 1.58E−04 | 2.41E−01 | 5.23−02 | 2.07E−03 | 3.85E−01 | 4.92E−01 | 2.54E−01 | 7.81E−01 |
| 12 | 1.98E−01 | 1.70E−01 | 3.64E−01 | 3.05E−01 | 6.94E−01 | 4.38E−01 | 6.38E−01 | 7.62E−01 | 5.84E−01 |
| 13 | 9.97E−05 | 3.71E−08 | 2.04E−01 | 2.50E−07 | 3.29E−03 | 4.03E−01 | 1.43E−01 | 3.13E−02 | 1.50E−02 |
| 14 | 1.51E−01 | 5.43E−02 | 8.32E−01 | 1.30E−03 | 3.05E−06 | 1.08E−01 | 1.60E−01 | 3.97E−01 | 8.99E−01 |
| 15 | 2.00E−02 | 5.00E−01 | 5.43E−01 | 9.82E−01 | 4.49E−02 | 2.36E−01 | 8.06E−01 | 8.46E−01 | 2.71E−01 |
| 16 | 1.03E−01 | 9.68E−01 | 6.50E−01 | 6.22E−03 | 2.61E−03 | 2.98E−01 | 1.76E−01 | 1.88E−01 | 6.51E−01 |
| 17 | 6.73E−02 | 9.60E−06 | 7.23E−01 | 9.82E−02 | 3.22E−03 | 6.62E−02 | 6.45E−01 | 1.36E−02 | 5.45E−01 |

As oxygen interacts with phosphate and ribose to influence the pH of the reaction mixture, it is not possible to interpret its effect individually. Therefore, the interactions of oxygen with phosphate and ribose are shown in FIG. 1C as a cubic plot.

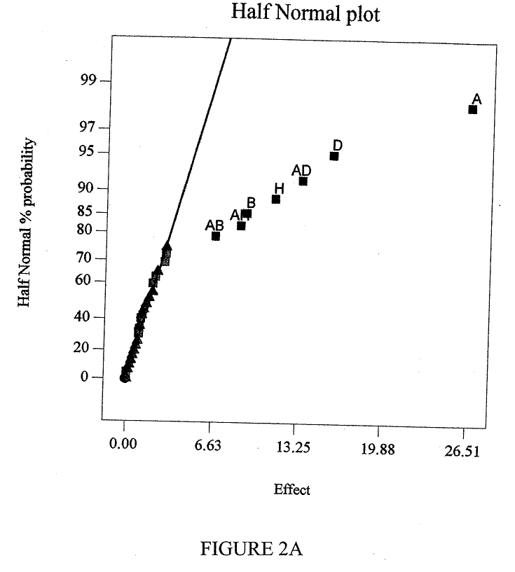

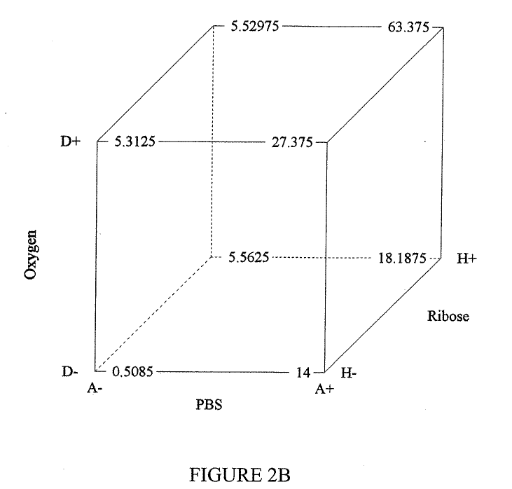

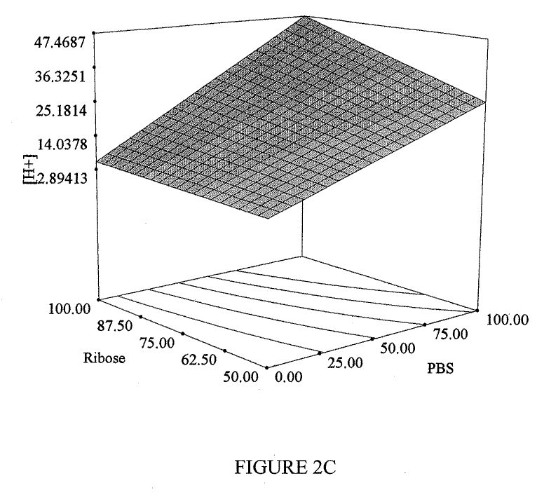

Effect of variables on protons formed: As the buffering capacity of the various runs (mixtures) were different, the change in pH was equated to the amount of H+ produced by using a calibration curve as described in the methods section, The values are based on titrating a 20 ml buffer solution. Run #8, which had all three buffers, produced the maximum amount of protons (73 mEq. corresponding to a ΔpH=−0.907). Minimum amount of H⁺ (0.01 mEq.) was produced in run #16 which had all the factors at the “low level” (see Table II). The half-normal % probability plot of the various factors and their two-factor interactions on the amount of protons generated is shown in FIG. 2A. Factors A, B, C, D, and H were significant in producing H⁺. Among the two factor interactions AB, AD, and AH were significant (see FIG. 2A). Table III, column 2 shows the statistical significance and R-Sqd. for the linear statistical model, and the significance of the main factors and their two-factor interactions. The estimate of coefficients for only the significant factors, in coded values, is shown in Table IV column 2. As A, D, H, AD, and AH were very significant, it was unclear if the influence of factor B on production of H⁺ was from Tris or from the three-factor interaction of ADH. Therefore, the data was reanalyzed, after deleting insignificant factors (factors C, E, F, and G), as a full three factorial design with phosphate, oxygen, and ribose, as well as a four factorial design that included Tris. From such analysis, we confirmed that the 3-factor interaction, ADH, influenced the formation of H⁺ rather than Tris. The p>F for the 3-factor linear model was 1.99E-15 and the R-Sqd. was 0.96. The main effect of phosphate, oxygen, and ribose along with their two and 3-factor interaction is shown in FIG. 2B as a cubic plot. The front left corner represents the amount of H⁺ (0.5085 mEq.) formed in the reaction mixture at the end of the incubation period when phosphate, oxygen, and ribose are at the low level.” The back right upper corner (63.37 mEq.) represents the effect when all three factors are present at their “high level.” The final equation for the production of protons, in coded form, is given by equation (2).
Δ[H+]=17.39+13.25*PBS+7.92*PO₂+5.68*ribose+6.72*PBS*PO₂+4.36*PBS*ribose+337*pO₂*ribose+4.58*PBS*pO₂*ribose (2)

FIG. 2C shows a combined contour and surface plot of the interaction between phosphate and ribose, mathematically computed, with pO₂ and EDTA at the “high level” and the rest of the factors at the “low level.”

Effect of variables on consumption of pO₂: Solutions that were purged with nitrogen for five minutes showed an average pO₂ of 55 mm of Hg, the rest had an average pO2 of 188 mm Hg. The change in pO₂ (mm of Hg) ranged from −117.7 to +41.7 with the mean being −36.12. Phosphate ions and probably oxygen were the only significant variables that affected the consumption of pO2. In general, the partial pressure of oxygen was noted to decrease at the end of the incubation period. In runs #2, 6, 9, and 13, the incubation mixture became anaerobic. The common features in these runs were the presence of phosphate at the “high level” and oxygen at the “low level.” In two runs (#7 and #16) there was a significant, consistent, and reproducible increase in the partial pressure of oxygen indicating that oxygen was formed during the incubation period. Runs #7 and #16 were significant for the absence of phosphates, light, and EDTA, and low levels of oxygen and a relatively low value in all the observed responses. The linear statistical model had a value of 1.43E-07 and an R-Sqd. of 0.82. There was no significant interaction between phosphate and oxygen in the consumption of oxygen. The linear model representing the consumption of oxygen in coded factors is given below.
−ΔpO₂=−36.12−27.37*PBS−12.63pO₂−8.52*ribose−8.04*EDTA (3)

Effect of variables on pCO₂: The partial pressure of carbon dioxide (pCO2) increased in all the runs. The maximum average increase of 21.6 mm was observed in run 8, and the least change (0.2 mm) was seen in run 16. The average increase in pCO₂ was 5.6 mm, In runs #8 and #16, all factors were at the “high level” and “low level” respectively. The statistical linear model was significant at p=5.50E-1 I and its R-sqd. was 0.9225 (see Table III).

Again, oxygen, phosphate, and their interaction had the greatest influence in the formation of carbon dioxide (p<1.00E-06). EDTA (p<0.0046) and Light (p<0.0309) were also noted to have an effect on production of carbon dioxide. Interestingly, ribose was not significant in influencing the pCO₂. The estimate of coefficients for significant factors is given in Table IV.

TABLE IV
THE ESTIMATE OF COEFFICIENTS OF FACTORS THAT SIGNIFICANTLY
INFLUENCE EACH RESPONSE

| | pH | H+ | PO2 | PCO2 | Fluore- | CBZ- | P1 | P2 | P3 |
|---|---|---|---|---|---|---|---|---|---|
| Intercept | −0.356 | 17.481 | −36.119 | 5.259 | 223.031 | 9.234 | 0.178 | 0.023 | 0.033 |
| A: PBS | −0.166 | 13.253 | −27.375 | 3.4781 | 189.612 | 5.833 | 0.171 | 0.017 | 0.026 |
| B: TRIS | | 1.581 | | | 30.263 | | | | |
| C: HEPPES | 0.112 | | | | | | | | |
| D: Oxygen | −0.156 | 7.917 | −12.63 | 3.666 | −28.375 | 2.373 | −0.054 | 0.020 | 0.032 |
| E: Light | | | | | 29.044 | | | | |
| F: Shaker | | | | | | | | | |
| G: EDTA | | | 1.266 | | 31.142 | | | | |
| H: Ribose | −0.121 | 5.682 | | | 45.325 | | | | |
| 11 | −0.104 | 3.372 | | | | | | | |
| 12 | | | | | | | | | |
| 13 | −0.088 | 6.724 | 3.434 | | | | | | |
| 14 | | | 1.572 | | 47.300 | | | | |
| 15 | | | | | | | | | |
| 16 | | | 1.272 | | | | | | |
| 17 | | 4.365 | | | | | | | |

The final equation of pCO₂ in terms of coded factors is shown in equation 4.
ΔpCO₂=±5.26+3.48*PBS±3.67*pO₂+1.27*EDTA+3.43*PBS*pO₂+1.57*PBS*light+1.27*PBS*EDTA (4)

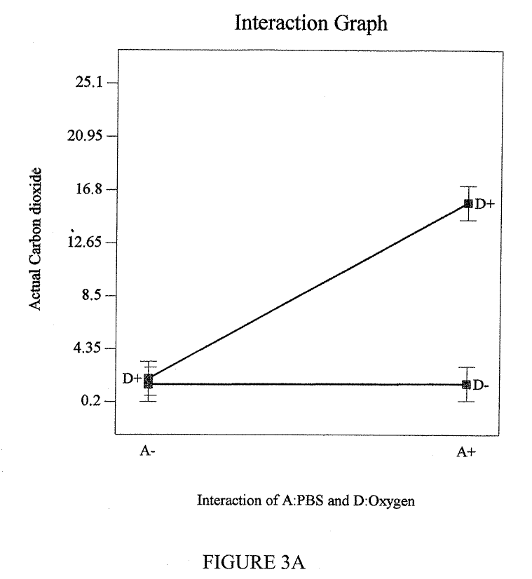

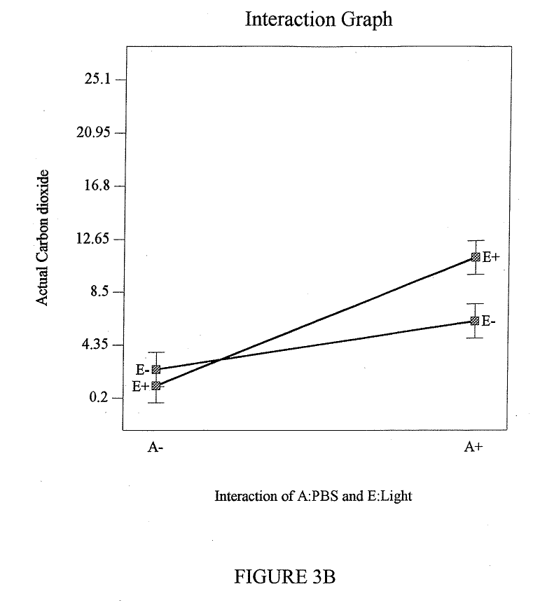

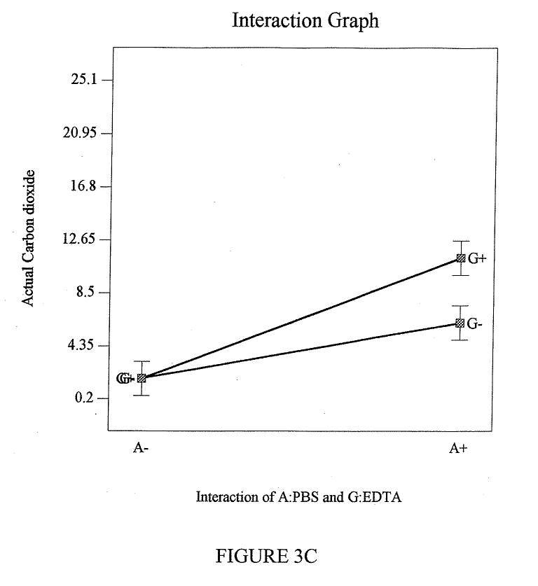

The two-factor interaction plots between phosphate and oxygen, light, and EDTA are shown FIGS. 3A, 3B, and 3C respectively.

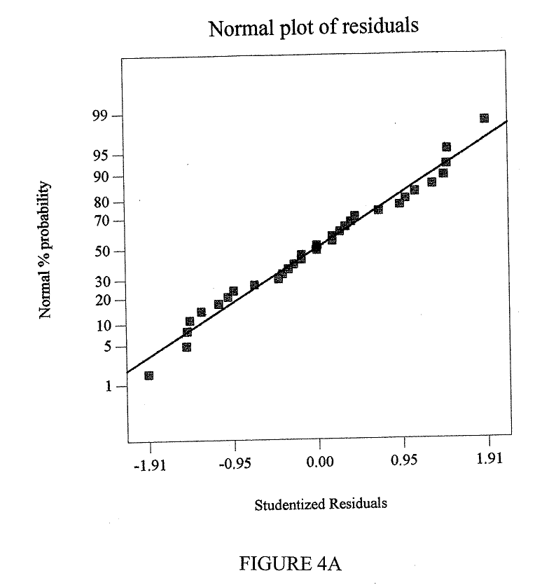

Effect of variables on fluorescence: The fluorescence response was influenced by many variables. Runs #6 and #8 fluoresced considerably while runs #1, #10, and #14 exhibited minimal fluorescence. The statistical linear model was significant at ‘p>F’ 5.60E-I2 with an R-Sqd. of 0.9819 (see Table III). FIG. 4A shows the normal % probability plot of the Studentized residuals. Table III shows the statistical significance of all the factors in the model; and Table IV shows the estimate of coefficients of significant factors only. Tris and oxygen showed a significant negative influence on fluorescence and shaker bath speed had the least effect. The rest of the factors exhibited varying degrees of significance. Interestingly, there was a significant interaction between phosphate-light, and phosphate-EDTA. FIG. 4B is a composite graph showing the effect of all the significant variables. FIG. 4C shows the interaction plot between phosphate and light. In the absence of phosphate light has minimum effect however, in the presence of phosphate light significantly increases fluorescence. It should be pointed out that the fluorescence studies were performed at only a single excitation/emission wavelength.
Fluor=223.03+169*PBS+47.3*PBS*light+45.33*ribose+31.14*EDTA−30.26*Tris+29.04*light−28.38*pO₂ (5)

Effect of variables on the consumption of CRZ-Lysine: The reaction consumed CBZ-lysine during the incubation period with the maximum consumption being 24% in run #8. Only phosphate, oxygen, and ribose significantly influenced this consumption. No interactions between variables were observed. The model was significant at p>5.40E-06 and the equation is given below
ΔCBZ-lysine=9.23+5.83*PBS+2.37*pO₂+2.24*ribose (6)

Effect of variables on the formation of products P1, P2, and P3: Three major products were identified based on HPLC analysis and labeled as P1, P2 and P3. Not all three products were present in all the runs and their amounts varied. The chemical identity of these products is not known. Phosphate influences the formation of all three products while oxygen is associated with form P2 and P3 only. PBS*O₂ interaction played a significant role in the formation of P2 and P3 while PBS*ribose influenced the formation of P2.

Effect of each variable on the responses: In the above section, the effect of variables on each response was summarized. Here we briefly summarize the effect of each variable on the responses. Only phosphate significantly influenced all responses and this trend was followed closely by oxygen. Tris decreased pO₂ and fluorescence formed. HEPES mainly increased pH and fluorescence. Ambient laboratory lighting increased pCO₂, fluorescence, and formation of P2. EDTA was associated with increase in pH, consumption of oxygen, formation of carbon dioxide, and fluorescence production. Ribose significantly altered pH, protons formation, oxygen consumption, and fluorescence production. Speed of the shaker did not influence on any of the responses investigated.

Among the factor interactions PBS-oxygen interaction influenced pH, H⁺, pCO₂, fluorescence, P2, and P3. PBS-light interaction affected formation of carbon-dioxide and fluorescence. PBS-EDTA interacted to enhance formation of carbon-dioxide and fluorescence. PBS-ribose interaction was associated with formation of protons, fluorescence, and P2.

Correlation of responses on each other: In the above paragraphs the effect of experimental variables on responses were described and here we report the relationship between responses. As there are eight responses, there are 36 combinations of response pairs. Correlation between responses is important to provide mechanistic insight in this complex reaction. In general, the amount of protons generated correlated better than pH with all the responses. [H⁺] was substantially associated (r-sqd>0.5) with consumption of oxygen and CBZ-lysine, and formation of CO₂, fluorescence, and P2. CBZ-lysine was strongly associated with fluorescence formation. P1 was also associated with fluorescence while P2 and P3 were not and P3 seems to be related to P2.

Discussion:

Statistical Model: Traditionally, one estimates the influence of experimental variables using the “one-factor-at-a-time” while keeping the other variables at some arbitrarily chosen constant value. In such a technique, one assumes the effect of a variable to be the same at all other variable levels and that the variables act only additively. In contrast, the factorial design helps determine the effect of a factor over a complete range of other variables, with greater precision, and accounts for nonadditive interactions. In general, initially examining a factor at two levels is efficient and economical, Therefore, to evaluate eight variables at two levels a full factorial design would consist of 2⁸ or 256 experiments. This technique elucidates the main effects and all of the possible two, three, and higher level interactions unambiguously. Although the full factorial design has considerable advantages over the one-factor-at-a-time, in practical terms, even the full factorial design has some redundancy. In terms of the magnitude generally, the main effects tend to be larger than the two-factor interactions, which in turn tends to be larger than the three factor interactions, and so on. The partitioning of each type of p-factor interactions in a 2^{f} design is given by the following equation:

$$\frac{f ! l ( f - p )}{p !}$$

Thus the 256 statistics is partitioned between 1 mean, 8 main effects, 28 2-factor interactions, 56 3-factor interactions, 70 4-factor interactions, 56 5-factor interactions, 28 6-factor interactions, 8 7-factor interactions, and 1 8-factor interaction, For quantitative continuous variables, as in our case, the higher order interactions tend to be negligible and can be disregarded. In addition, in a higher order full factorial design of five or more variables, all variables may not be significant. In other words, some variables would be relatively insignificant and simply add to the “noise” of the system. Thus, only a fraction of the original lull factorial run of experiments will contain the “signal.” Fractional factorial designs have the ability to extract the “signal” efficiently in fewer experiments by taking advantage of this redundancy. Thus, fractional factorial designs are excellent for screening a large number of variables. As our initial goal was to identify the effect of the main factors independent of other main effects, and their two-factor we chose a resolution IV fractional factorial design. This design uses only 1/16 of the full factor design, the main effects are independent of each other but confounded with seven 3-factor interactions, and the two factor interactions are confounded with three other 2-factor interactions. We assumed that not all the factors would be significant for all the responses. Therefore, after deleting the insignificant factors the data could be reevaluated less ambiguously. In addition, one could use chemistry insight to verify statistical results.

If the effects represent a sample from a normal distribution, we would expect to see them form a straight line on a % normal probability plot of the effects. Usually, only a few effects turn out to be important which show up as outliers on the % normal probability plot. The % half-normal probability plot follows the same principle as the full % normal probability plot except that the sign of the effects is ignored in plotting. Thus, large absolute values show up as outliers in the upper right-hand corner of the graph. The line for the half-normal plot should start from the origin and go through the set of near-zero effects. The effect of factors on each response was analyzed using both probability plots. H In the half-normal probability plot, insignificant effect will be near zero, scattered in a normal distribution. That is, significant effects fall to the right of the line and away from zero. Replicates provide an independent estimate of pure experimental variability or error over the design space. The pure error estimate is used for evaluation of statistical significance. On a normal probability plot, pure error points should fall on a line coincident with insignificant effects.

Mechanistic Model:

As mentioned in the introduction section, our initial intention was two folds, first to determine which of the experimental variables maximally influenced the NEG reaction under in vitro physiological conditions and second to determine the causes of irreproducibility of cited literature, Based on conventional wisdom that sustained hyperglycemia is strongly associated with long-term complications, we anticipated ribose to be the most significant variable influencing fluorescence. We were also aware that buffers influence the extent of glycation under in vitro conditions and that phosphate buffer reacts poorly with radicals while organic buffers such as Tris and HEPES scavenge reactive intermediates. In addition, phosphates had a greater effect glycation, among the buffers. The effect of phosphate has been traditionally attributed to the presence of adventitious trace metals that can catalyze this reaction even at micromolar concentrations. Thus, the use of metal chelators has been advocated to inhibit the catalytic effect of trace metals. We therefore anticipated EDTA to considerably reduce fluorescence. As the reaction is not diffusion controlled, we did not expect the speed of the shaker to have any effect nor did we anticipate low-level (50 ft/candles) of ambient laboratory light to affect any response. As indicated in the results section, the chemometric analysis revealed several interesting features. Phosphates significantly modulate this reaction more than excess ribose under the present experimental conditions. There is a considerable change in pH of the solution in spite of being buffered. Experiments that had normal tension of oxygen at the beginning of the reaction were rendered anaerobic with high pCO₂. In some cases in which the solution was purged with nitrogen, the reaction produced oxygen. EDTA did not inhibit fluorescence formation. Also, several interactions between variables were observed and trace metal analysis indicated that CBZ-lysine was the main source of adventitious trace metals, Based on these observations we believe that the sources of irreproducibility arise from lack of experimental details to the dead space in the container, the partial pressure of oxygen, the amount of adventitious trace metals in the reagents, the intensity and duration of ambient laboratory light. In the following paragraphs, we have attempted to explain some of our observations based on information obtained from the literature.

Acid forming reactions: Among the variables phosphate, oxygen, and ribose strongly affected the formation of protons; and among the responses, the consumption of CBZ-lysine and formation of carbon dioxide were associated with production of acidity. This suggests that acidic group may result from the oxidation of ribose, consumption of CBZ-lysine. HCl with liberation of HCl, and formation of carbon dioxide.

D-ribose, in solution, exists in six tautomeric forms: α- and β-pyranose, α- and β-furanose, the open chain aldehyde, and its hydrated form. The β-pyranose form predominates in solution at 37 C and pH 7.0 (Cortes, 1991). The ratio of the hydrate to aldehyde in solution is approximately 10:1 (Angyal, 1984) and the two forms represent less than 1% of the tautomers. Ribose like other a-hydroxy aldehydes, forms an enediol anion in neutral or basic conditions. The enediol anion is an essential intermediate in the Lobry de Bruyn-Alberda van Ekenstein transformations give hydroxy-carboxylates. Only the acyclic form enolizes and may undergo β-elimination, direct oxidation by oxygen, or concomitant degradation, e.g., C—O or —C—C bond fission. Smith and Thornalley observed that the enolization of monosaccharides and its subsequent oxidation to ketoaldehyde, an α-dicarbonyl, in presence of air is associated with hydrogen peroxide. In principle, hydrogen peroxide detected during the formation of dicarbonyl sugars may be consumed in other oxidative reactions. For example, α-hydroxy acids and α-keto acids are cleaved by hydrogen peroxide with a-hydroxy acids giving aldehydes or ketones, and α-keto acids generating acids (March pg. 1087). This degradation step by hydrogen peroxide, of ahydroxy acids, when catalyzed by iron (FeSO₄) is known as Ruff degradation and is used extensively to elucidate the structure of sugars.

In addition to ribose, other chemical intermediates such as Schiff-base, carbinolamine, and Amadori product could be oxidized or generate acidic by-products (Stadtman, 1952; Katchalsky, 1953). Ahnmed et al, (Ahmed, 1986) from their studies on glycated proteins and fructosyl-lysine a model Amadori product, have identified N-carboxymethyllysine (CML) and erythronic acid as a product of glycoxidation; the reaction requires oxygen and trace metals catalyze it. Analogous reaction with ribose-CBZ-lysine would also result in generation of protons and a decrease in pH. Scheme I shows the above possible reactions that may produce acidic groups.

Effect of oxygen: The initial oxygen pressure in solutions that were not purged with nitrogen varied. The solubility of oxygen in pure water is given by Benson and Krause (Benson, 1982) and its solubility in aqueous solutions containing different ions is given by

$$log ( He / {He}_{0} ) = {0.5 \sum {Hiz}_{i}^{2} {C}_{i}}_{i}$$

Where He, He₀ are the Henry coefficient in aqueous ionic solution and pure water respectively. Henry coefficient is the ratio of the partial pressure of a gas and its equilibrium concentration in the liquid, Hi is the salting-out parameter, z the ion charge, and C the concentration of species i. As the solutions had different concentration of buffers and no attempt was made to keep the ionic strength of the reaction constant, the initial partial pressure of oxygen varied by about 10%.

The reactivity of oxygen is characterized by its electronic configuration. Oxygen has considerable affinity for electrons, as evidenced by the fact that most oxidation reactions involving oxygen are exothermic. Molecular oxygen, or dioxygen, under ambient conditions is kinetically stable. The origin of this stability is due to the distribution of two obits valence electrons in a ground-state triplet (T0), 3 Σg- (unpaired), in which the electrons are distributed in the degenerate pair of antibonding r*-orbitals. As the two electrons are in planes 90° apart, dioxygen behaves as a biradical and may only participate in singlepoint attack radical-like reactions. Since most organic molecules usually have paired electrons and are in singlet ground states, their reaction with dioxygen triplet ground-state C, is spin forbidden. This kinetic sluggishness can be circumvented by one of three ways: 1) reacting the triplet with a radical to form peroxyl radical 2) complexing with a paramagnetic metal ion, or 3) exciting the triplet to one of its two low-lying singlet states (Si and S2). In S1 (1Δg) the electron spins are paired and may lie parallel or perpendicular to each other leaving the other π* “empty.” Due to this positional variation, the S1 oxygen can participate as a single-point radical or in concerted addition reactions with a two-point attack, or as an electrophile via its empty orbital. In S2 state and energetically higher of the two (1 Σg+), the electrons are forced to be coplanar in spatial disposition, and could be expected to participate only in two-point concerted addition reactions. The first two pathways are not spin restricted, and the third pathway usually occurs photochemically. Under biological conditions, oxygen is reduced to superoxide, hydrogen peroxide, hydroxyl radical and water via a 4-electron reduction. Transition metals facilitate this reduction process. The overall reaction may be written as
4Fe(II)+O₂+4H⁺→4Fe(III)+2H₂O

Under our experimental conditions the consumption of oxygen is significantly influenced by phosphates, (see Table III) and is associated with formation of carbon dioxide and consumption of CBZ-lysine (see Table V). However, ambient laboratory light had no significant effect. Thus, the consumption of oxygen may be largely proceeding via triplet state rather than as a singlet oxygen with the reaction being initiated by trace or adventitious transition metal and later by free radicals generated during the reaction. Under conditions simulating physiological environment, Spoehr (Spoehr, 1934) observed that oxygen reacts with trioses, in the absence of amino acids but in the presence of sodium ferropyrophosphate-phosphate mixtures, to produce carbon dioxide. Clinton observed that phosphate or arsenate catalyze the reaction, and the rate depends on concentration of salt present, but not as much on pH (Clinton, 1937). Traube (Traube, W. 1936) observed that salts of copper, cobalt, iron, and nickel also catalyzed the consumption of oxygen formatting carbon dioxide and formic acid. Oxygen may also be consumed in oxidizing the Amadori product for example in the formation of carboxymethyl-CBZ-lysine (Scheme II). In two different experiments (runs #7 & 16) pO₂ was greater at the end of the incubation period. In other words oxygen was formed. Runs #7 and #16 were significant for the absence of phosphates, light, and EDTA, and low levels of oxygen. Trace metals are present in CBZ-lysine probably as a complex, and if the redox potential is significantly decreased the system may oscillate with water being oxidized and oxygen being reduced.

Formation of Carbon dioxide: As mentioned previously, one source of carbon dioxide is the oxidation of ribose. However, in the presence of amino acids. Stadtman (Stadtman, 1952) has shown that the major source of carbon dioxide is from the carboxyl group of amino acid via Strecker type degradation. Carbon dioxide is also formed during oxidation of amino-acids by Fenton reagents (H₂O₂+Fe⁺⁺)(Stadtman, 1991). We also noted that EDTA, and ambient laboratory light in the presence of phosphates, influenced the formation of carbon dioxide, The hydroxyalkyl radicals formed may also undergo dehydration & decarboxylation reaction as suggested by Steenken (Steenken, 1973). Davies (Davies, 1996) has recently shown that Fe₂-EDTA alkoxy radical can give rise to C-terminal decarboxylation with the release of CO₂ radical. Vasquez-Vivar (Vasquez-Vivar, 1997) observed that keto-carboxylic acids might undergo both a one or two electron oxidative decarboxylation by Fe/EDTA and hydrogen peroxide respectively.

Fluorescence formation: Non-enzymatic glycation reaction gives rise to fluorescent products. In vivo, the extent of fluorescence formation in tissues with long half-life, such as collagen, is generally observed to be proportional to age except in diabetic patients. In diabetes, a disease characterized by hyperglycemia, the extent of collagen fluorescence is considerably higher than in age matched non-diabetic population reflecting the increased accumulation of advanced glycation end (AGE) products. In our study, fluorescence, formation was increased by phosphate, ribose, light, EDTA, and interaction between phosphate-light. The effect of phosphate on NEG and fluorescence formation is described in detail below. Oxygen decreased fluorescence probably by quenching. The interaction of phosphate and light suggests the participation of radicals.

Consumption of CSZ-Lysine and formation of P1, P2, & P3: Consumption of CBZ-lysine was associated with phosphate, oxygen, and ribose (Table III) and with acidity, fluorescence, and P2 formation (Table V). From this, one may infer that CBZ-lysine may be used up in more than one pathway with glycoxidation being the predominant one generating acidity and fluorescence with P2 may be a product of this reaction.

Effect of phosphate: Phosphate, as an inorganic buffer salt or as a phosphate-ester of reducing sugar, accelerates Maillard reaction by facilitating enolization. Other anionic buffers such as arsenate and carbonate also catalyze this reaction (Spoehr 1934). These anions catalyze the formation of 1-2 enediol tautomer and stabilize it by forming adducts. Amino acids, cyanides (Robertson, 1981), osmium tetraoxide, and phenolate ions are also known to form adducts with enediols.

The autoxidation of reducing sugars occurs rapidly under alkaline conditions (DeWit, 1979). Hough (Hough, 1967) and Thornalley et al. (Thornalley, 1984}observed the rate of autoxidation of monosaccharides to be in the order of phosphates>HEPES>Tris under physiological conditions. They attributed this to the ability of the monosaccharides to form enediol in the same order. Wolff and Hunt (Wolff, 1991; Wolff 1989) in their investigation of glucose autoxidation and protein modification, observed that increasing concentration of phosphate produced a proportionally increased amount of ketoaldehyde as well as glycation of protein. Watkins et al., (Watkins, 1987) observed that the kinetics of glycation of RNAase, lysozome, cytochrome, and hemoglobin were substantially faster in phosphate as compared to cationic buffers. Based on kinetic investigation of glycation they concluded that phosphates facilitate the Amadori rearrangement. Basic proteins like RNAase, lysozome, and cytochrome c, were glycated more than acidic proteins like albumin and Mb.

Borsook and Wastcnenys (Borsook, 1925) and Schwimmer (Schwimmer, 1953) observed that phosphated sugars such as, glucose 6-phosphate and fructose 6-phosphate browned more rapidly than glucose and fructose. They further noticed that phosphate buffer salt considerably accelerated this browning process. Kato (Kato, 1956) and Webb (Webb, 1935) reported that phosphates enhance the overall rate of browning or fluorescence. Burton et al; (Burton, 1963) also observed similar enhancement of glucose glycine reaction by phosphate; the presence of trace amounts of iron also accelerated the Maillard reaction.

In spite of numerous examples of the enhancement of various stages of Maillard reaction by phosphates, its catalytic effect has been undermined as transition metals also exhibit similar effect (Burton, 1963) (Markuze, 1963). Micromolar amounts of iron, the levels usually present in most buffer solutions, are enough to induce autoxidation physiological pH (Buettner, 1988). In all the above cases there is no clear experimental evidence that care was taken to thoroughly remove trace metals from reagents nor was the amount of trace metals in such reagents determined.

In face of this controversy, our data suggests that phosphates may significantly and independently influence the Maillard reaction. In our experiment, the greatest source of trace metals, on a molar basis, was CBZ-lysine present in all the experiments. Thus, the effect of buffers was obtained against approximately the same amount of trace metals. Other supportive evidence that phosphates play an important role are as follows. Iron is not soluble in water at pH 7.4. They may exist at that pH as a mixed aquo-chelated complex at physiological pH. The redox potential of the Fe(III)/Fe(II) is high and is significantly changed by ligands (see Table V). Wang (Wang, 1978), de Figueiredo (de Figuciredo 1981), and Bobbio (Bobbio, 1973) observed that copper accelerated browning of glucose-glycine in a buffer-free system considerably decreased from 500% at pH 3 to 21% at pH 6.2, however, in presence of phosphates at 6.5 the reaction was considerably enhanced (Markuze, 1963). Phosphates are also known to catalyze the oxidation of ferrous ion by oxygen (Kurimura, 1969; Tadolini, 1987; Biaglow, 1997) as well as the reduction of ferric ions by reducing agents (Goto, 1970; Mitra, 1985) at neutral pH. Reinke (Reinke, 1994) have postulated the primary oxidant formed during Fe²⁺ autoxidation in phosphate to be an iron-oxygen-phosphate complex such as a ferryl species. Similarly, fenyl-O₂-EDTA complex have been proposed as intermediate in the reaction of Fe/EDTA reaction with H₂O₂ (Rush, 1986). Taborsky (Taborsky, 1972) observed that phosphate ions strongly influenced the reaction between ferrous ions and cytochrome c. Fransson (Fransson, 1996) observed that phosphates, free of metal contaminants, influenced the oxidation of methionine in the presence of oxygen via formation of phosphorylated sulfonium ion formation. They also observed a significant interaction between phosphate and visible light and suggested the participation of free radicals. Reinke (Reinke, 1995) observed that inorganic phosphates markedly influenced the rates of free radical formation in liver microsomes. Thus, in biological medium, the ligand gives the trace metal its catalytical properties.

Effect of Tris and Good's buffer: Tris had minimum effect on pH or production of carbon dioxide, Interestingly, it also decreased fluorescence probably by quenching hydroxy radical. Shiraishi et al. (Shiraishi, 1993) observed that interactions of hydroxyl radicals with Tris and Good's buffer may produce formaldehyde. Adhikari and Tappell (Adhikari, 1973) have reported that polyvalent cation complex with melanoidins, resulting in diminished florescence; and perhaps Tris behaves similarly. Hopes and Tris are also known to form secondary radicals by effectively scavenging oxygen radicals from the iron catalyzed Huber-Weiss process and hydrogen peroxide (Murphy, 1974; Halliwell, 1986; Grady, 1998; Saprin, 1998; Burkitt, 1991).

Effect of Light: Bohart (Bohart, 1995) observed that light had a pronounced effect on Maillard products under certain conditions. When samples of a solution of D-glucose-glycine were sealed under nitrogen, those stored at 50° C. in laboratory illumination became darker than those kept in dark. However, when partially browned solutions under air or oxygen were exposed to light their color gradually bleached. Mizutari et al. (Mizutari, 1997) observed higher levels of CML in sun-exposed areas than sun-unexposed areas of the skin.

Effect of EDTA on responses: Transition metals, characterized by their incomplete d-orbitals, have the ability to rearrange their electronic configuration. For example, in Fe(II) one such rearranged state is the intermediate spin (S=1) with two unpaired electrons. Once this reorganization has occurred coupling between the unpaired electrons on both dioxygen ground state (³Σ_{g}⁻) and iron takes place resulting in the formation of iron dioxygen bond (McClure, 1960). The reaction between dioxygen and Fe(II) may also occur with electronic reorganization within the dioxygen singlet states. In biological medium oxygen is reduced to water through a four electron reduction process with the concomitant consumption of two equivalents of protons. Trace metals may easily donate the electrons during this process and thus influence the out come of this reaction. This step completes the oxidation cycle. In presence of reducing agents, the trace metal is reduced to its initial oxidation state and the cycle is completed with trace metals acting as a catalyst. The above 2±7 set of reactions is shown in scheme IV. The rate at which trace metal ions accept or donate their electrons is governed by redox potentials, concentration of ions and substrates, and the size of the ligand with smaller ligands favoring faster reaction rates. The redox potential for a given metal ion is strongly influenced by the type of ligand, their orientation, and electron-delocalization onto the ligand. Examples of some one-electron redox couples of Fe(II)/Fe(III) and its complexes including EDTA is given in Table VI, For iron to catalyze the formation of oxygen reactive species in biological medium requires the availability of at least one coordination site that is open or occupied by a readily dissociable ligand such as water (Graf, 1984). The site also needs to be free of steric hindrance so that either oxygen species or a reducing sugar can be accommodated in the complex to facilitate the transfer of electrons. As most chelators decrease the redox potential and thereby facilitate the electron transfer process, their inhibitory characteristics usually stem from their steric features. Wolff and Dean (Wolff, 1987) in their study of glucose autoxidation observed that trace metal chelators exhibited a peculiar biphasic response in which increasing concentration of chelators showed decreasing inhibitory effect on attachment of glucose to BSA. They attributed this behavior as evidence for two different pathways for glucose attachment, one dependent on trace metals and the other independent of metal. We believe that the later pathway represents the general acid-base catalytic effect of polyanion buffers.

The importance of amino acids, polyanions, and transition metals is their ability to catalyze the enolization reaction at physiological pH (Shallenberger, 1984) and facilitate the transfer of electron from the radical anion to oxygen. In nitrogen nucleophilic reactions, the carbonyl carbon of open-chain form of sugar reacts with an amino group to form a carbinol-amine intermediate. In presence of excess reactants, the carbinol-amine intermediate can dehydrate to form a Schiff base. If the reducing sugar is an aldose, the Schiff base under acidic conditions rearranges to 1,2-eneaminol through a sigmatropic shift, which later ketonizes to an Amadori product. Reversible reactions generate the carbinol-amine, Schiff base (Hayashi, 1986) (Namiki, 1975) and the Amadori products (Hodge, 1953). The Amadori product, a ketoamine (Yaylayan, 1994), through a series of substitution, rearrangement, and dehydration steps, give rise to highly reactive dicarbonyl sugar compounds that culminate in forming the final (AGE) products. Pentosidine and pyralanes are some of the products found in-vivo and used as biomarkers for the NEG reaction. The original NEG mechanism did not emphasize oxidation or free radical involvement but focused more on the formation of Amadori products that altered surface charge, hydrogen bonding capability, cellular recognition, and formed complex products capable of cross-linking (Pongor, 1984; Brownlee, 1994; Harding, 1985).

### EXAMPLE 2

This example illustrates the relationship of serum phosphate levels, serum glucose levels and glycation as indicated by HbA1c levels in humans.

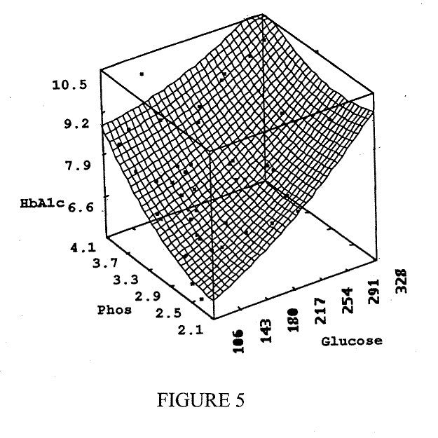

We performed a retrospective chart review of blood data of our veterans at the Veterans Medical Center stored in the computer database. The samples were restricted to morning (before 8:00 am) blood draw data that included HbA1c, serum phosphate, glucose, over a four-month period. Only patients with normal inorganic phosphate levels (2.0-5.0 mg/dl) were used for the study and our results are shown as a three-dimensional plot in FIG. 5. The x-axis and y-axis represents average phosphate and glucose level over a four-month period and the z-axis represents the HbA1c values. As one would expect the extent of HbA1c level increases with increasing average serum glucose. However, it is interesting to note that for any given value of glucose, the value of HbA1c increases with increasing levels of serum phosphate. This is a non-linear effect with the interaction more pronounced at lower concentrations of glucose and phosphate than at higher. Thus, the trend observed in vitro is also observed in vivo that is, HbA1c is modulated by factors other than reducing sugars and phosphate is one such factor.

All references cited in this specification are hereby incorporated by reference. Any discussion of references cited herein is intended merely to summarize the assertions made by their authors and no admission is made that any reference or portion thereof constitutes relevant prior art. Applicants reserve the right to challenge the accuracy and pertinency of the cited references.

### REFERENCES

1. V. M. Monnier, M. Glomb, A. Eigawish, D R. Sell, Diabetes 45 Suppl 3, S67 (1996).
2. B. J. Ortwerth, S M. Slight, M. Prabhakaram, Y. Sun, J. B. Smith, Biochimica et Biophysica Acta 1117, 207 (1992).
3. A. Papoulis, Y. al-Abed, R. Bucala, Biochemistry 34,648 (1995).
4. A. T. Lee, A. Cerami, Progress in Clinical & Biological Research 304, 291 (1989).
5. R. Ikan, The Maillard Reaction Consequences for the chemical and life sciences, R. Ikan, Ed. (John Wiley & Sons, New York, 1996), p. 161.
6. V. M. Monnier, Journal of Gerontology 45, B105 (1990).
7. V. M. Monnier, Progress in Clinical & Biological Research 304, 1 (1989).
8. V. M. Monnier, A. Cerami, Science 211, 491 (1981).
9. P. J. Thornalley, Environmental Health Perspectives 64, 297 (1985).
10. S. P. Wolff, Z. A. Bascal, J. V., Hunt, Progress in Clinical & Biological Research 304, 259 (1989).
11. M. Namiki, 1. Hayashi, J. Agric. Food Chem. 23, 487 (1975).
12. T. H˜ayashi, M. Namiki, Amino-carbonyl reactions in food and biological systems, E. Fujimori, M. Namiki and H. Kato, Eds. (Elsevier, Amsterdam, 1986), p. 29.
13. I. Sakari, K. Sugioka, M. Nakano, Biochim. Biophysics. Acta 1043, 27 (1990).
14. K. J. Wells-Knecht, D V. Zyzak, J. E. Litchfield, S. R. Thorpe, J. W. Baynes, Biochemistry 34, 3702 (1995).
15. T. Miyata, et al, Nephrology, Dialysis, Transplantation 11 Suppl 5, 27 (1996).
16. V. M. Monnier, et al, Nephrology, Dialysis, Transplantation 11 Suppl 5, 20 (1996).
17. T. Miyata, K. Maeda, K. Kurokawa, C. van Ypersele de Strihou, Nephrology, Dialysis, Transplantation 12, 255 (1997).
18. C. A. Colaco, M. D. Ledesma, C R. Harrington, J. Avila, Nephrology, Dialysis, Transplantation 11 Suppl 5, 7 (1996).
19. J. Thome, et al, Nervenar:t 67, 924 (1996).
20. T. Kimura, et al, Neuroreport 6, 866 (1995).
21. M. A. Smith, et al, Annals of the New York Academy of Sciences 738, 447 (1994).
22. T. Kimura, et al, Neuroscience Letters 219, 95 (1996).
23. G. E. P. Box, W. G. Hunter, J. S. Hunter, Statistics for Experimenters An introduction to design, data analysis, and model building (John Wiley & Sons, Inc.; New York, 1978).
24. S. J. Cortes, T. L. Mega, R. L. van Etten, J. Org. Chem. 56, 943 (1991).
25. P. R. Smith, P. J. Thornalley, Biochemistry International 28, 429 (1992).
26. F. M. Stadtman, C O. Chichester, Mackinney O., Journal of the American Chemical Society 74, 3194 (1952).
27. 27. A. Katchaisky, N. Sharon, Biochim. Biophysics. Acta 10,290 (1953).
28. M. U. Ahmed, S. R. Thorpe, J. W. Baynes, Journal of Biological Chemistry 261, 4889 (1986).
29. B. B. Benson, D. Krause, J. Chem. Phys. 64, 689 (1982).
30. H. A. Spoehr, H. W. Mimer, J. Am. Chem. Soc 56,2068 (1934).
31. M. Clinton, R. S. Hubbard, Journal of Biological Chemistry 119, 467 (1937).
32. E. R. Stadtman, B. S. Berlett, J. Biol. Chem. 266, 17201 (1991).
33. S. Steenken, Schultze-Frohlidine, Tetrahedron Lett. 653 (1973).
34. M. J. Davies, Archives of Biochemistry & Biophysics 336, 163 (1996).
35. J. Vasquez-Vivar, A. Denicola, R. Radi, O. Augusto, Chemical Research in Toxicology 10 (7), 786 (1997).
36. P. Robertson, S. E. Fridovich, H. P. Misra, 1. Fridovich, Archives of Biochemistry & Biophysics 207-2, 282 (1981).
37. L. Bough, A. C. Richardson, 1967), p. 274.
38. P. Thornalley, S. Wolff, J. Crabbe, A. Stem, Biochimica et Biophysica Acta 797, 276 (1984).
39. J. V. Hunt, S. P. Wolff, Free Radical Research Communications 12-13 Pt 1, 115 (1991).
40. N. O. Watkins, C T. Neglia-Fisher, D. O. Dyer, S. R. Thorpe, J. W. Baynes, Journal of Biological Chemistry 262, 7207 (1987).
41. H. Borsook, H. Wasteneys, Biochemistry Journal 19, 1128 (1925).
42. S. Schwimmer, H. S. Olcott, Journal of the American Chemical Society 75, 4855 (1953).
43. H. Kato, Bull. Agr. Chem. Soc. Japan 20 supp 1, 273 (1956).
44. B. H. Webb, J. Dairy Science 2,81 (1935).
45. H. S. Burton, D. J. McWeeney, D. O. Biltcliffe, J. Food Sci. 28, 631 (1963).
46. Z. Markuze, Roczniki Panstwowego Zakladu Hig. 14, 65 (1963).
47. G. R. Buettner, J. Biochem. Biophys. Methods 16, 27 (1988).
48. S M. Wang, P A. Bobbio, F. O. Bobblo, An. Acad. Bras. Cienc. 50,55 (1978).
49. F. O. Bobbio, P. A. Bobbio, L. M. V. Trevisan, Lebensm. Wiss. Technol. 6,215 (1973).
50. Y. Kurimura, Bull. Chem. Soc. Jpn. 42 (8), 2238 (1969).
51. B. Tadolini, A. M. Sechi, Free Radical Res. Commun. 4 (3), 161 (1987).
52. J. E. Biaglow, Kachur, A. V., Radiation Res 142 (2), 181 (1997).
53. K. Goto, H. Tamura, M. Nagayama, Inorganic Chemistry 9, 963 (1970).
54. Mitra A. K., M L. Matthews, International Journal of Pharmaceutics 23, 185 (1985).
55. L. A. Reinke, J. M. Rau, P B. McCay, FreeRadical Biology & Medicine 16 (4), 485 (1994).
56. J. D. Rush, W. H. Koppenol, J. Biol. Chem. 261 (15), 6730 (1986).
57. O. Taborsky, Biochemistry 11 (5), 729 (1972).
58. L. A. Reinke, D R. Moore, J. R. M. P. B. Rau, Archives of Biochemistry & Biophysics 316, 758 (1995).
59. H. Shiraishi, M. kataoka, Y. Morita, J. Umemoto, Free Radical Res. Commun. 0.19(5), 315 (1993).
60. H. R. Adhikari, A L. Tappel, J. Food Sci. 38, 486 (1973).
61. P. A. Murphy, J. S. Lin, H. S. Olcott, Archives of Biochemistry & Biophysics 164, 776 (1974).
62. B. Halliwell, J. M. Gutteridge, Archives of Biochemistry & Biophysics 246, 501 (1986).
63. A M. Saprin, L. H. Piette, Archives of Biochemistry & Biophysics 180, 480 (1998).
64. M. J. Burkitt, B. C. Gilbert, Free Radical Res. Commun. 14, 107 (1991).
65. K. Mizutari, T. Ono, K. Ikeda, K. Kayashima, S. Horiuchi, Journal of Investigative Dermatology 108, 797 (1997).
66. D. S. McClure, Radiation Res. suppl 2, 218 (1960).
67. E. Graf, J. R. Mahoney, R. G. Bryant, J. W. Eaton, J. Biol. Chem. 259(6), 3620 (1984).
68. S. P. Wolff, R. I. Dean, Biochemical Journal 245, 243 (1987).

69. R. S. Shallenberger, Food Chem. 15, 1 (1984).

70. J. E. Hodge, J. Agric. Food Chem. 1,928 (1953).

71. V. A. Yaylayan, A. Huyghues-Despointes, Critical Reviews in Food Science & Nutrition 34, 321 (1994).
72. S. Pongor, P. C. Ulrich, F. A. Bencsath, A. Cerami, Proceedings of the National Academy of Sciences of the United States of America 81, 2684 (1984).
73. M. Brownlee, Diabetes 43, 836 (1994).
74. J. J. Harding, Adv. Prot. Chem. 37, 247 (1985).
75. K. Kunika, M. Itakura, K. Yamashita, Life Sciences 45, 623 (1989).
76. K. Kunika, M. Itakura, K. Yamashita, Diabetes Research and Clinical Practice 13, 37 (1991).
77. K. Kunika, K. Yamashita, M. Itakura, Diabetes Research and Clinical Practice 17,9 (1992).
78. D R. Sell, V. M. Monnier, J. Clin. Invest 85, 380 (1990).
79. D R. Sell, et al., Diabetes Metabolism Reviews 7,239 (1991).
80. P. Odetti, J. Fogarty, D R. Sell, V. M. Monnier, Diabetes 41, 153 (1992).
81. J. Sabater, et al, Am. J. Nephrol. 11,370991).
82. R. J. Haley, D. M. Ward, Am. J. Kidney Dis. 8, (2), 115 (1986).
83. Anonymous, Alzheimer Disease (1994).
84. M. P. Vitek, et al, Proceedings of the National Academy of Sciences of the United States of America 91 (11), 4766 (1994).
85. A. Takedo, et al., Neuroscience Letters 221(1), 17 (1996).
86. G. Lee, N. Cowan, M. Kirschner, Science 239, 285 (1988).
87. S. C. Seldon, T. D. Pollard, J. Biol Chem 258, 7064 (1983).
88. M. Butler, M L. Shelanski, J. Neurochem 47, 1517 (1986).
89. V. M. Lee, B. J. Balm, L. Otvos, Q. Irojanowski, Science 251,675 (1991).
90. M. Abdel-Ghany, A. K. El-Sebae, D. Shalloway, J. Biol Chem 268 (16), 11976 (1993).
91. S. S. Krishnan, D. R. McLachlan, B. Krishnan, S. S. Fenton, J. E. Harrison, Sci. Total Environ. 71 (1), 59 (1988).
92. H. Yoshida, F. Yoshimasu, Nippon Rinsho 54 (1), 111 (1996).
93. Nishida Y, Ito 5, Z. Naturforsch 50 (7-8), 571 (1995).
94. W. R. Mundy, T M. Freudenrich, P. R. Kodavanti, Mol. Chem. Neuropathol. 32 (1-3), 41 (1997).
95. S. Toda, Y. Yase, Biol. Trace Elem. Res. 61 (2), 207 (1998).
96. T. Iarkka, N. Yli-Mayiy, R. M. Mannermaa, K. O. J. Majamaa, Biochim. Biophysics. Acta 1180 (3), 294 (1993).
97. M. D. Ledesma, Bonay, P, C. A. Colaco, J. Avila, J. Biol Chem 269 (34), 21614 (1994).
98. S. D. Yan, et al., Nat. Med 1 (7), 693 (1995).
   The present application includes the following aspects:
   1. A method for treating a disease involving non-enzymatic glycation, the method comprising administering to a patient in need thereof an effective amount of at least one agent that decreases serum phosphate concentration, wherein prior to treatment the patient has a serum phosphate concentration of not more than about 5.0 mg/dL.
   2. A method of Aspect 1, wherein the at least one agent that decreases serum phosphate concentration is a diuretic having carbonic anhydrase activity, a phosphate binder or a combination thereof.
   3. A method of Aspect 2, wherein the diuretic is acetazolamide, dichlorphenamide, methazolamide, furosemide or a combination thereof.
   4. A method of Aspect 2, wherein the at least one agent that decreases serum phosphate is aluminum hydroxide, calcium hydroxide, a calcium salt, magnesium hydroxide, a magnesium salt, lanthanum carbonate, a phosphate binding cationic polymer or a combination thereof.
   5. A method of Aspect 4, wherein the calcium salt calcium acetate, calcium carbonate, calcium gluconate or a combination thereof.
   6. A method of Aspect 4, wherein the magnesium salt is magnesium acetate, magnesium carbonate, magnesium gluconate or a combination thereof.
   7. A method of Aspect 4, wherein the phosphate binder is lanthanum carbonate.
   8. A method of Aspect 4, wherein the phosphate binding cationic polymer is sevelamer hydrochloride.
   9. A method of Aspect 1, wherein the patient suffers from diabetes or diabetic complications, renal failure, Alzheimers disease, Pick's disease, decreased skin elasticity, male erectile dysfunction, pulmonary fibrosis, atherosclerosis, and ocular diseases.
   10. A method of Aspect 1, wherein the patient is at risk for mortality and other adverse cardiovascular events following acute myocardial infarction or following percutaneous coronary intervention.
   11. A method of Aspect 1, wherein the patient has increased levels of glycation products.
   12. A method of Aspect 11, wherein the patient has HbA1c levels of 6% or greater.
   13. A method of Aspect 1, wherein the at least one agent is administered in an amount effective in decreasing serum phosphate concentration to not less than about 2 mg/dL.
   14. A method of Aspect 1, further comprising administering an effective amount of at least one agent that decreases blood glucose levels, wherein the at least one agent that decreases blood glucose levels is insulin, a sulfonylurea, a biguanide compound, a meglitinide compound or a compound that acts upon glucose digestion or metabolism.
   15. A method for treating a disease involving non-enzymatic glycation, the method comprising selecting at least one agent on the basis of the agent being effective in decreasing serum phosphate concentration and administering an effective amount of the at least one agent to a patient in need thereof, wherein prior to treatment the patient has a serum phosphate concentration of not more than about 5.0 mg/dL.
   16. A method of Aspect 15, wherein the at least one agent that decreases serum phosphate concentration is a diuretic having carbonic anhydrase activity, a phosphate binder or a combination thereof.
   17. A method of Aspect 16, wherein the diuretic is acetazolamide, dichlorphenamide, methazolamide, furosemide or a combination thereof.
   18. A method of Aspect 16, wherein the at least one agent that decreases serum phosphate is aluminum hydroxide, calcium hydroxide, a calcium salt, magnesium hydroxide, a magnesium salt, lanthanum carbonate, a phosphate binding cationic polymer or a combination thereof.
   19. A method of Aspect 18, wherein the calcium salt calcium acetate, calcium carbonate, calcium gluconate or a combination thereof.
   20. A method of Aspect 18, wherein the magnesium salt is magnesium acetate, magnesium carbonate, magnesium gluconate or a combination thereof.
   21. A method of Aspect 18, wherein the phosphate binder is lanthanum carbonate.
   22. A method of Aspect 18, wherein the phosphate binding cationic polymer is sevelamer hydrochloride.
   23. A method of Aspect 15, wherein the patient suffers from diabetes or diabetic complications, renal failure, Alzheimers disease, Pick's disease, decreased skin elasticity, male erectile dysfunction, pulmonary fibrosis, atherosclerosis, and ocular diseases.
   24. A method of Aspect 15, wherein the patient is at risk for mortality and other adverse cardiovascular events following acute myocardial infarction or following percutaneous coronary intervention.
   25. A method of Aspect 15, wherein the patient has increased levels of glycation products.
   26. A method of Aspect 25, wherein the patient has HbA1c levels of 6% or greater.
   27. A method of Aspect 15, wherein the at least one agent is administered in an amount effective in decreasing serum phosphate concentration to not less than about 2 mg/dL.
   28. A method of Aspect 15, further comprising administering an effective amount of at least one agent that decreases blood glucose levels, wherein the at least one agent that decreases blood glucose levels is insulin, a sulfonylurea, a biguanide compound, a meglitinide compound or a compound that acts upon glucose digestion or metabolism.
   29. A method for treating a disease involving non-enzymatic glycation, the method comprising administering to a patient in need thereof an effective amount of at least one agent that decreases serum phosphate concentration, wherein the patient suffers from a disease other than end stage renal disease.
   30. A method of Aspect 29, wherein the at least one agent that decreases serum phosphate concentration is a diuretic having carbonic anhydrase activity, a phosphate binder or a combination thereof.
   31. A method of Aspect 30, wherein the diuretic is acetazolamide, dichlorphenamide, methazolamide, furosemide or a combination thereof.
   32. A method of Aspect 30, wherein the at least one agent that decreases serum phosphate is aluminum hydroxide, calcium hydroxide, a calcium salt, magnesium hydroxide, a magnesium salt, lanthanum carbonate, a phosphate binding cationic polymer or a combination thereof.
   33. A method of Aspect 32, wherein the calcium salt calcium acetate, calcium carbonate, calcium gluconate or a combination thereof.
   34. A method of Aspect 32, wherein the magnesium salt is magnesium acetate, magnesium carbonate, magnesium gluconate or a combination thereof.
   35. A method of Aspect 32, wherein the phosphate binder is lanthanum carbonate.
   36. A method of Aspect 32, wherein the phosphate binding cationic polymer is sevelamer hydrochloride.
   37. A method of Aspect 29, wherein the patient suffers from diabetes or diabetic complications, renal failure, Alzheimers disease, Pick's disease, decreased skin elasticity, male erectile dysfunction, pulmonary fibrosis, atherosclerosis, and ocular diseases.
   38. A method of Aspect 29, wherein the patient is at risk for mortality and other adverse cardiovascular events following acute myocardial infarction or following percutaneous coronary intervention.
   39. A method of Aspect 29, wherein the patient has increased levels of glycation products.
   40. A method of Aspect 39, wherein the patient has HbA1c levels of 6% or greater.
   41. A method of Aspect 29, wherein the at least one agent is administered in an amount effective in decreasing serum phosphate concentration to not less than about 2 mg/dL.
   42. A method of Aspect 29, further comprising administering an effective amount of at least one agent that decreases blood glucose levels, wherein the at least one agent that decreases blood glucose levels is insulin, a sulfonylurea, a biguanide compound, a meglitinide compound or a compound that acts upon glucose digestion or metabolism.
   43. A method for treating a disease involving non-enzymatic glycation, the method comprising selecting at least one agent on the basis of the agent being effective in decreasing serum phosphate concentration and administering an effective amount of the at least one agent to a patient in need thereof, wherein the patient suffers from a disease other than end stage renal disease.
   44. A method of Aspect 43, wherein the at least one agent that decreases serum phosphate concentration is a diuretic having carbonic anhydrase activity, a phosphate binder or a combination thereof.
   45. A method of Aspect 44, wherein the diuretic is acetazolamide, dichlorphenamide, methazolamide, furosemide or a combination thereof
   46. A method of Aspect 44, wherein the at least one agent that decreases serum phosphate is aluminum hydroxide, calcium hydroxide, a calcium salt, magnesium hydroxide, a magnesium salt, lanthanum carbonate, a phosphate binding cationic polymer or a combination thereof.
   47. A method of Aspect 46, wherein the calcium salt calcium acetate, calcium carbonate, calcium gluconate or a combination thereof.
   48. A method of Aspect 46, wherein the magnesium salt is magnesium acetate, magnesium carbonate, magnesium gluconate or a combination thereof.
   49. A method of Aspect 46, wherein the phosphate binder is lanthanum carbonate.
   50. A method of Aspect 46, wherein the phosphate binding cationic polymer is sevelamer hydrochloride.
   51. A method of Aspect 43, wherein the patient suffers from diabetes or diabetic complications, renal failure, Alzheimers disease, Pick's disease, decreased skin elasticity, male erectile dysfunction, pulmonary fibrosis, atherosclerosis, and ocular diseases.
   52. A method of Aspect 43, wherein the patient is at risk for mortality and other adverse cardiovascular events following acute myocardial infarction or following percutaneous coronary intervention.
   53. A method of Aspect 43, wherein the patient has increased levels of glycation products.
   54. A method of Aspect 53, wherein the patient has HbA1c levels of 6% or greater.
   55. A method of Aspect 43, wherein the at least one agent is administered in an amount effective in decreasing serum phosphate concentration to not less than about 2 mg/dL.
   56. A method of Aspect 43, further comprising administering an effective amount of at least one agent that decreases blood glucose levels, wherein the at least one agent that decreases blood glucose levels is insulin, a sulfonylurea, a biguanide compound, a meglitinide compound or a compound that acts upon glucose digestion or metabolism.
   57. A method of preventing or diminishing non-enzymatic glycation in a patient, the method comprising administering to a patient in need thereof, an effective amount of at least one agent that decreases serum phosphate concentration, wherein prior to administering the agent, the patient has a serum phosphate concentration of not more than about 5.0 mg/dL.
   58. A method of Aspect 57, wherein the at least one agent that decreases serum phosphate concentration is a diuretic having carbonic anhydrase activity, a phosphate binder or a combination thereof.
   59. A method of Aspect 58, wherein the diuretic is acetazolamide, dichlorphenamide, methazolamide, furosemide or a combination thereof.
   60. A method of Aspect 58, wherein the at least one agent that decreases serum phosphate is aluminum hydroxide, calcium hydroxide, a calcium salt, magnesium hydroxide, a magnesium salt, lanthanum carbonate, a phosphate binding cationic polymer or a combination thereof.
   61. A method of Aspect 60, wherein the calcium salt calcium acetate, calcium carbonate, calcium gluconate or a combination thereof.
   62. A method of Aspect 60, wherein the magnesium salt is magnesium acetate, magnesium carbonate, magnesium gluconate or a combination thereof.
   63. A method of Aspect 60, wherein the phosphate binder is lanthanum carbonate.
   64. A method of Aspect 60, wherein the phosphate binding cationic polymer is sevelamer hydrochloride.
   65. A method of Aspect 57, wherein the patient suffers from diabetes or diabetic complications, renal failure, Alzheimers disease, Pick's disease, decreased skin elasticity, male erectile dysfunction, pulmonary fibrosis, atherosclerosis, and ocular diseases.
   66. A method of Aspect 57, wherein the patient is at risk for mortality and other adverse cardiovascular events following acute myocardial infarction or following percutaneous coronary intervention.
   67. A method of Aspect 57, wherein the patient has increased levels of glycation products.
   68. A method of Aspect 67, wherein the patient has HbA1c levels of 6% or greater.
   69. A method of Aspect 57, wherein the at least one agent is administered in an amount effective in decreasing serum phosphate concentration to not less than about 2 mg/dL.
   70. A method of Aspect 57, further comprising administering an effective amount of at least one agent that decreases blood glucose levels, wherein the at least one agent that decreases blood glucose levels is insulin, a sulfonylurea, a biguanide compound, a meglitinide compound or a compound that acts upon glucose digestion or metabolism.
   71. A method of preventing or diminishing non-enzymatic glycation in a patient, the method comprising selecting at least one agent on the basis of the agent being effective in decreasing serum phosphate concentration and administering an effective amount of the at least one agent to a patient in need thereof wherein prior to treatment the patient has a serum phosphate concentration of not more than about 5.0 mg/dL.
   72. A method of Aspect 71, wherein the at least one agent that decreases serum phosphate concentration is a diuretic having carbonic anhydrase activity, a phosphate binder or a combination thereof.
   73. A method of Aspect 72, wherein the diuretic is acetazolamide, dichlorphenamide, methazolamide, furosemide or a combination thereof.
   74. A method of Aspect 72, wherein the at least one agent that decreases serum phosphate is aluminum hydroxide, calcium hydroxide, a calcium salt, magnesium hydroxide, a magnesium salt, lanthanum carbonate, a phosphate binding cationic polymer or a combination thereof.
   75. A method of Aspect 74, wherein the calcium salt calcium acetate, calcium carbonate, calcium gluconate or a combination thereof.
   76. A method of Aspect 74, wherein the magnesium salt is magnesium acetate, magnesium carbonate, magnesium gluconate or a combination thereof.
   77. A method of Aspect 74, wherein the phosphate binder is lanthanum carbonate.
   78. A method of Aspect 74, wherein the phosphate binding cationic polymer is sevelamer hydrochloride.
   79. A method of Aspect 71, wherein the patient suffers from diabetes or diabetic complications, renal failure, Alzheimers disease, Pick's disease, decreased skin elasticity, male erectile dysfunction, pulmonary fibrosis, atherosclerosis, and ocular diseases.
   80. A method of Aspect 71, wherein the patient is at risk for mortality and other adverse cardiovascular events following acute myocardial infarction or following percutaneous coronary intervention.
   81. A method of Aspect 71, wherein the patient has increased levels of glycation products.
   82. A method of Aspect 81, wherein the patient has HbA1c levels of 6% or greater.
   83. A method of Aspect 71, wherein the at least one agent is administered in an amount effective in decreasing serum phosphate concentration to not less than about 2 mg/dL.
   84. A method of Aspect 71, further comprising administering an effective amount of at least one agent that decreases blood glucose levels, wherein the at least one agent that decreases blood glucose levels is insulin, a sulfonylurea, a biguanide compound, a meglitinide compound or a compound that acts upon glucose digestion or metabolism.
   85. A pharmaceutical composition for treating a disease involving non-enzymatic glycation, the composition comprising an effective amount of at least one agent that decreases serum phosphate concentration and an effective amount of at least one agent that decreases serum glucose levels.
   86. A composition of Aspect 85, wherein the at least one agent that decreases serum phosphate concentration is a diuretic having carbonic anhydrase activity, a phosphate binder or a combination thereof.
   87. A composition of Aspect 86, wherein the diuretic is acetazolamide, dichlorphenamide, methazolamide, furosemide or a combination thereof.
   88. A composition of Aspect 86, wherein the at least one agent that decreases serum phosphate is a phosphate binder selected from the group consisting of aluminum hydroxide, calcium hydroxide, a calcium salt, magnesium hydroxide, a magnesium salt, lanthanum carbonate, a phosphate binding cationic polymer and a combination thereof.
   89. A composition of Aspect 88, wherein the calcium salt calcium acetate, calcium carbonate, calcium gluconate or a combination thereof.
   90. A composition of Aspect 88, wherein the magnesium salt is magnesium acetate, magnesium carbonate, magnesium gluconate or a combination thereof.
   91. A composition of Aspect 88, wherein the phosphate binder is lanthanum carbonate.
   92. A composition of Aspect 88, wherein the phosphate binding cationic polymer is sevelamer hydrochloride.
   93. A composition of Aspect 85, wherein the at least one agent that decreases blood glucose levels is insulin, a sulfonylurea, a biguanide compound, a meglitinide compound or a compound that acts upon glucose digestion or metabolism.
   94. A kit for treating a disease involving non-enzymatic glycation, the kit comprising an effective amount of at least one agent that decreases serum phosphate concentration and an effective amount of at least one agent that decreases serum glucose levels packaged separately in a container.
   95. A kit of Aspect 94, wherein the at least one agent that decreases serum phosphate concentration is a diuretic having carbonic anhydrase activity, a phosphate binder or a combination thereof.
   96. A kit of Aspect 95, wherein the diuretic is acetazolamide, dichlorphenamide, methazolamide, furosemide or a combination thereof.
   97. A kit of Aspect 95, wherein the at least one agent that decreases serum phosphate is a phosphate binder selected from the group consisting of aluminum hydroxide, calcium hydroxide, a calcium salt, magnesium hydroxide, a magnesium salt, lanthanum carbonate, a phosphate binding cationic polymer and a combination thereof.
   98. A kit of Aspect 97, wherein the calcium salt calcium acetate, calcium carbonate, calcium gluconate or a combination thereof.
   99. A kit of Aspect 97, wherein the magnesium salt is magnesium acetate, magnesium carbonate, magnesium gluconate or a combination thereof.
   100. A kit of Aspect 97, wherein the phosphate binder is lanthanum carbonate.
   101. A kit of Aspect 97, wherein the phosphate binding cationic polymer is sevelamer hydrochloride.
   102. A kit of Aspect 94, wherein the at least one agent that decreases blood glucose levels is insulin, a sulfonylurea, a biguanide compound, a meglitinide compound or a compound that acts upon glucose digestion or metabolism.

What is claimed is:

1. A method for treating a disease involving non-enzymatic glycation, the method comprising administering to a patient in need thereof an effective amount of at least one agent that decreases serum phosphate concentration, wherein prior to treatment the patient has a serum phosphate concentration of not more than about 5.0 mg/dL.

2. A method of claim 1, wherein the at least one agent that decreases serum phosphate concentration is a diuretic having carbonic anhydrase activity, a phosphate binder or a combination thereof.

3. A method of claim 2, wherein the diuretic is acetazolamide, dichlorphenamide, methazolamide, furosemide or a combination thereof.

4. A method of claim 2, wherein the at least one agent that decreases serum phosphate is aluminum hydroxide, calcium hydroxide, a calcium salt, magnesium hydroxide, a magnesium salt, lanthanum carbonate, a phosphate binding cationic polymer or a combination thereof.

5. A method of claim 4, wherein the calcium salt calcium acetate, calcium carbonate, calcium gluconate or a combination thereof.

6. A method of claim 4, wherein the magnesium salt is magnesium acetate, magnesium carbonate, magnesium gluconate or a combination thereof.

7. A method of claim 4, wherein the phosphate binder is lanthanum carbonate.

8. A method of claim 4, wherein the phosphate binding cationic polymer is sevelamer hydrochloride.

9. A method of claim 1, wherein the patient suffers from a disease selected from the group consisting of diabetes, diabetic complications, renal failure, Alzheimers disease, Pick's disease, decreased skin elasticity, male erectile dysfunction, pulmonary fibrosis, atherosclerosis, and an ocular disease.

10. A method of claim 1, wherein the patient is at risk for mortality and other adverse cardiovascular events following acute myocardial infarction or following percutaneous coronary intervention.

11. A method of claim 1, wherein the patient has increased levels of glycation products.

12. A method of claim 11, wherein the patient has HbA1c levels of 6% or greater.

13. A method of claim 1, wherein the at least one agent is administered in an amount effective in decreasing serum phosphate concentration to not less than about 2 mg/dL.

14. A method of claim 1, further comprising administering an effective amount of at least one agent that decreases blood glucose levels, wherein the at least one agent that decreases blood glucose levels is insulin, a sulfonylurea, a biguanide compound, a meglitinide compound or a compound that acts upon glucose digestion or metabolism.

15. A method of preventing or diminishing non-enzymatic glycation in a patient, the method comprising administering to a patient in need thereof, an effective amount of at least one agent that decreases serum phosphate concentration, wherein prior to administering the agent, the patient has a serum phosphate concentration of not more than about 5.0 mg/dL.

16. A method of claim 15, wherein the at least one agent that decreases serum phosphate concentration is a diuretic having carbonic anhydrase activity, a phosphate binder or a combination thereof.

17. A pharmaceutical composition for treating a disease involving non-enzymatic glycation, the composition comprising an effective amount of at least one agent that decreases serum phosphate concentration and an effective amount of at least one agent that decreases serum glucose levels.

18. A composition of claim 17, wherein the at least one agent that decreases serum phosphate concentration is a diuretic having carbonic anhydrase activity, a phosphate binder or a combination thereof.

19. A composition of claim 18, wherein the diuretic is acetazolamide, dichlorphenamide, methazolamide, furosemide or a combination thereof.

20. A composition of claim 18, wherein the at least one agent that decreases serum phosphate is a phosphate binder selected from the group consisting of aluminum hydroxide, calcium hydroxide, a calcium salt, magnesium hydroxide, a magnesium salt, lanthanum carbonate, a phosphate binding cationic polymer and a combination thereof.
